(19) United States
(12) Patent Application Publication
(10) Pub. No.: US 2026/0137471 A1
(43) Pub. Date: May 21, 2026

(54) SURGICAL SYSTEM HAPTIC FEEDBACK SYSTEMS AND METHODS

(71) Applicant: Intuitive Surgical Operations, Inc., Sunnyvale, CA (US)

(72) Inventor: Ashwinram SURESH, San Jose, CA (US)

(73) Assignee: Intuitive Surgical Operations, Inc., Sunnyvale, CA (US)

(21) Appl. No.: 19/119,532

(22) PCT Filed: Oct. 11, 2023
(86) PCT No.: PCT/US2023/034912

§ 371 Date: Apr. 9, 2025

Related U.S. Application Data
(60) Provisional application No. 63/415,487, filed on Oct. 12, 2022.

(51) Int. Cl. A61B 34/00; A61B 34/35; B25J 9/16; B25J 13/02

(52) U.S. Cl. CPC A61B 34/76; A61B 34/35; B25J 9/1689; B25J 13/025

(57) ABSTRACT

Systems and methods are provided for control of a surgical system. Haptic feedback is provided to an input device of the surgical system. A moment at a reference location of a distal end portion of an instrument of the surgical system is determined. A deflection of the reference location is then determined based on the moment. On a first condition in which the deflection is greater than a deflection threshold, an indication is provided to an operator of the input device that a restriction of the haptic feedback is provided to, or is available to be provided to, the input device.

### CROSS-REFERENCE TO RELATED APPLICATIONS

This application claims priority to and the filing date benefit of U.S. Provisional Patent Application No. 63/415,487, entitled “Surgical System Haptic Feedback Systems and Methods,” filed Oct. 12, 2022, the disclosure of which is incorporated herein by reference in its entirety.

### BACKGROUND

The embodiments described herein relate to surgical systems, and more specifically to teleoperated surgical systems that operate with at least partial computer assistance. More particularly, the embodiments described herein relate to systems and methods for determining a deflection of a medical instrument in order to control a surgical system that includes force feedback provided to a human system operator.

Known techniques for Minimally Invasive Surgery (MIS) employ instruments to manipulate tissue that can be either manually controlled or controlled via hand-held or mechanically grounded teleoperated medical systems that operate with at least partial computer-assistance (“telesurgical systems”). Many known MIS instruments include a therapeutic or diagnostic end effector (e.g., forceps, a cutting tool, or a cauterizing tool) mounted on an optional wrist mechanism at the distal end of a shaft. During an MIS procedure, the end effector, wrist mechanism, and the distal end of the shaft are typically inserted into a small incision or a natural orifice of a patient to position the end effector at a work site within the patient's body. The optional wrist mechanism can be used to change the end effector's position and orientation with reference to the shaft to perform a desired procedure at the work site. In known instruments, motion of the instrument as a whole provides mechanical degrees of freedom (DOFs) for movement of the end effector, and the wrist mechanisms generally provide the desired DOFs for movement of the end effector with reference to the shaft of the instrument. For example, for forceps or other grasping tools, known wrist mechanisms are able to change the pitch and yaw of the end effector with reference to the shaft. A wrist may optionally provide a roll DOF for the end effector, or the roll DOF may be implemented by rolling the shaft. An end effector may optionally have additional mechanical DOFs, such as grip or knife blade motion. In some instances, wrist and end effector mechanical DOFs may be combined. For example, U.S. Pat. No. 5,792,135 (filed May 16, 1997) discloses a mechanism in which wrist and end effector grip DOFs are combined.

Force sensing surgical instruments are known and together with associated telesurgical systems deliver haptic feedback to a surgeon performing an MIS procedure. The haptic feedback may increase the immersion, realism, and intuitiveness of the procedure for the surgeon. For effective haptics rendering and accuracy, force sensors may be placed on a medical instrument and as close to the anatomical tissue interaction as possible. One approach is to include a force sensor unit having electrical sensor elements (e.g., strain sensors or strain gauges) at a distal end of a medical instrument shaft to measure strain imparted to the medical instrument. The measured strain can be used to determine the force imparted to the medical instrument and as input from which the desired haptic feedback may be generated.

FIG. 1A shows one example of a known force sensor unit that includes a cantilever beam 810 attached between the instrument distal tip component 510 (e.g., in some cases a clevis or other wrist or end effector component) and the instrument shaft 410 that extends back to the mechanical structure. As illustrated, strain sensors 830 are coupled to the beam to measure strain in X- and Y-directions as shown (arbitrary Cartesian directions that are orthogonal to each other and to a longitudinal axis of the beam and instrument shaft). For example, the strain sensors can optionally include full Wheatstone bridges (full bridges). In some cases, in order to reject common modes, such as temperature, the strain sensors are each split into two sets, with one set on the distal end of the beam and the other set on the proximal end of the beam. Because the beam is secured to a distal portion of the instrument shaft, the strain sensors sense strain on the beam orthogonal to a longitudinal axis AA of the shaft. A force component F_{A} (FIG. 1B) applied orthogonal to the beam (i.e., a force in an X-Y plane, such as an X or Y force) is determined by subtracting strain measurements determined by the full-bridges at the proximal and distal end portions of that side face of the beam.

During the employment of the medical instrument, however, certain operating conditions may be encountered under which the output of the force sensor unit may not accurately indicate the force imparted to the medical instrument. The operating conditions may, for example, correspond to the positioning of the medical instrument, an operation being performed by the medical instrument, and/or a fault condition. The inaccuracies that may be encountered may limit the ability of the telesurgical system to deliver accurate haptic feedback to the surgeon performing the procedure.

For example, in certain positions, the strain indicated by the strain sensors may be less than the strain that would be imparted to the medical instrument in response to the applied force F_{A} affecting the distal tip component 510 when not in the certain positions. More specifically, some known force sensing medical instruments may include or be used with a substantially stiff structure 901 that at least partially surrounds the beam 810 and upon contact either stops beam 810's further deflection or effectively changes beam 810's stiffness and resulting deflection characteristics. For example, some known force sensing medical instruments may include a protective structure (e.g., shroud) that covers the strain sensors 830 and their associated wires during use. In other words, the structure 901 is a structure that does not deflect to the same degree as the beam 810. To ensure the beam 810 remains cantilevered for accurate force sensing, the structure 901 may not be directly coupled to the distal tip component 510. Instead, the structure 901 may be separate from the distal tip component to allow the beam to deflect when affected by the applied force F_{A} (see FIG. 1B). In certain situations, however, the distal end of the structure 901 may contact the beam (or a portion of the medical instrument surrounding the beam) or the distal tip component, thereby limiting deflection of the beam. FIG. 1B shows one example, in which the beam 810 is deflected in the X direction such that it contacts one side of the distal end of the structure 901 (e.g., the shroud), which limits or prevents further bending of the beam 810 in the X direction by an amount that is dependent upon structure 901's rigidity and the relative stiffeness between the structure 901 and the beam 810.

Although limiting the displacement of the beam can advantageously prevent overload of the beam 810 and/or the strain sensors 830, we have discovered that such known systems that engage the beam at a single point can cause a change in the strain distribution over the length of the beam 810. In other words, the beam 810 no longer functions as a cantilevered beam anchored solely at one end. The contact location between structure 901 and beam 810 acts as a fulcrum around which beam 810 bends. As a result, the strain sensors 830 produce signals that do not accurately represent the applied force F_{A} (e.g., the actual force affecting the medical instrument). Specifically, we have discovered that in certain situations the contact between the distal end of the structure 901 and the beam 810 may cause distortion of the signals produced by the strain sensors 830. In certain situations, the distortion can cause the force sensed by the strain sensors 830 to be in the opposite direction of the applied force F_{A} (this phenomenon can be referred to as “force inversion” because a human operator's haptic sensation of force direction based on the erroneous strain sensor signals will be inverted from the correct force direction).

FIGS. 2A and 2B are rigid body mechanics diagrams of example known force sensing medical instrument of FIGS. 1A and 1B to further illustrate this example of force distortion and inversion. As shown in FIG. 2A, the contact between the shroud and the beam can be modeled as a single point contact (at GND 2). In FIG. 2A, the distance L represents the distance from the base of the beam 810 (point GND 1) to the point where the shroud (e.g., the substantially stiff structure 901) contacts the beam 810 (point GND 2). The distance D represents the distance between the point where the shroud contacts the beam 810 (point GND 2) and location at which the applied force F_{A} is applied to or by the distal tip component 510.

FIG. 2B is a rigid body mechanics diagram of the beam showing exaggerated deflection of the beam as a result of the contact at point GND 2. As shown, we have discovered that the strain distribution along the top surface of the beam transitions from a proximal region of compression to a more distal region of tension, which causes the signals from the strain sensors 830 to inaccurately represent the applied force F_{A}.

FIG. 2C shows the modeled forces with the beam “cut” at point GND 2 for purposes of analyzing the force and pure moment of the beam. FIG. 2C shows the reactive force F_{R} produced by the single point contact, the consolidated force F (such as may be indicated by the strain sensors 830 of FIG. 1B), and a pure moment (M) (e.g., a couple) produced by the opposing force vectors of equal magnitude according to rigid body mechanics. By modeling the beam at the point of contact (at GND 2), the additional deflection (i.e., beyond this point of contact away from the shaft) can be considered as zero. Using the static and deflection equations shows that there are two different strain profiles over the entire beam length. The strain profile (8) on the top side of the beam for the beam length l being between 0 and L is given by Eq. (1), where E is the modulus of elasticity of the beam, I is the moment of inertia of the XY cross section of the beam, and r is the perpendicular distance of the strain gauge from the neutral axis of the beam:

$$ℰ ( l ) = - \frac{F ( L - l ) r}{EI} + \frac{Mr}{EI} \qquad Eq . ( 1 )$$

The strain profile (ε) on the top side of the beam for the beam length l being between L and L+D is given by Eq. (2):

$$ℰ ( l ) = - \frac{{F}_{A} ( L + D - l ) r}{EI} \qquad Eq . ( 2 )$$

Thus, at certain locations along the beam 810, the strain sensors 830 produce signals indicative of the force F and not necessarily the applied force F_{A}. The signal associated with force F includes force components associated with both the applied force F_{A} and the reactive force F_{R}. This results in a distortion (and even an inversion of force direction) of the determined force relative to the applied force F_{A} that is actually exerted on the beam.

FIG. 3A is a graph showing the strain along the top of the beam 810 along the length of the beam based on Eq. (1) and Eq. (2) for the condition when the beam 810 substantially contacts the structure 901 at the single point of contact (GND 2). To further illustrate force distortion, FIG. 3B is a graph showing measured force (e.g., a determined force based on the strain signals) as a function of the actual force applied. As shown, when the beam 810 is not in contact with the shroud (e.g., the substantially stiff structure 901), for example, when the actual force applied does not cause sufficient bending of the beam 810 to result in the displacement of the beam 810 being affected by the shroud, the relationship between the measured force (e.g., force measurements derived from strain gauges) and the applied force (e.g., actual force component in the XY plane) is linear, which allows for an accurate calibration (i.e., based on the slope of the line). At conditions in which the beam 810 is in contact with the shroud (as shown in FIG. 1B and illustrated in FIG. 2B), however, the measured force decreases as the actual force increases. It should be appreciated that force distortion is not limited to sensors using strain gauges and that any force sensor technology that is implemented on a similar cantilever beam architecture can experience force distortion.

When the measured force is used to produce haptic feedback to a person operating an instrument that includes the beam (e.g., haptic feedback at an input device the person is using to control the instrument), this measured force distortion/force inversion problem can result in an undesirable positive feedback loop, which could cause unexpected or undesirable movement at the input device. This discovery is more fully described in in U.S. Patent Publication No. US 2021/0353373 (filed May 17, 2021), entitled “Hard Stop that Produces a Reactive Moment Upon Engagement for Cantilever-Based Force Sensing,” which is incorporated herein by reference in its entirety for all purposes.

In view of this situation, the art is continuously seeking new and improved systems and methods for control of a surgical system.

### SUMMARY

This summary introduces certain aspects of the embodiments described herein to provide a basic understanding. This summary is not an extensive overview of the inventive subject matter.

The present disclosure includes systems and methods that facilitate the provision of haptic feedback to an input unit of a surgical system under restricted feedback and unrestricted feedback conditions. Accordingly, the systems and methods disclosed herein can be employed to determine whether the deflection of a portion of medical instrument of the surgical system is sufficient to necessitate the implementation of the restricted feedback condition. The magnitude of the deflection is determined based on a determined moment at a reference location.

In one aspect, the present disclosure is directed to a method of control for a surgical system. The surgical system includes a controller, an input device, and a medical instrument. The medical instrument is operably coupled to the input device via the controller. The controller translates operator inputs to the input device into movements and/or operations of the medical instrument. The method includes, providing, via the controller, haptic feedback to the input device. The haptic feedback provides the operator of the system (e.g., the surgeon) with kinesthetic inputs that represent of the forces encountered by the medical instrument. The controller also determines a moment at a reference location of a distal end portion of the instrument. A deflection of the reference location is then determined based on the moment. On a condition in which the determined deflection is larger than a deflection threshold, the controller provides an indication to an operator of the input device that a restriction of the haptic feedback is provided to, or is available to be provided to, the input device. In the restriction of the haptic feedback can include limited, filtered, revised, and/or modelled feedback that is provided to the input device automatically or in response to a user selection.

In some embodiments, the reference location is located at a longitudinal position along the medical instrument that is coplanar with a hard stop location that limits deflection of the reference location.

In some embodiments, the medical instrument includes a force sensor unit and an end effector coupled to the distal end portion of the instrument. The force sensor unit includes a beam and one or more strain sensors coupled to the beam. The moment is determined based on output of the strain sensor(s).

In some embodiments, the strain sensor(s) include a first bridge circuit and a second bridge circuit. The first bridge circuit includes a first strain gauge resistor and a second strain gauge resistor. The second bridge circuit includes a third strain gauge resistor and a fourth strain gauge resistor. The moment is determined based on the output voltage of the first bridge circuit and the output voltage of the second bridge circuit.

In some embodiments, the controller determines an estimated applied force on the medical instrument based on the moment and the determined force. The controller then executes an operation of the surgical system based on the estimated applied force.

For example, in some embodiments, the magnitude of the haptic feedback delivered to the operator of the input device is based on the estimated applied force. As an additional example, in some embodiments, the controller halts an operation of the surgical system if the estimated applied force exceeds a threshold value.

In some embodiments, a surgical system includes an input device, a controller, and a medical instrument that is supported by a manipulator unit. The medical instrument is operably coupled to the input device. The controller is operably coupled to the manipulator unit and the input device. The controller includes at least one processor and a haptic feedback module that are configured to perform multiple operations. The operations include providing haptic feedback to the input device, determining a moment at a reference location of a distal end portion of the instrument, and determining a deflection of the reference location based on the moment. On a first condition in which the deflection is larger than a deflection threshold, an indication is provided to an operator of the input device that a restriction of the haptic feedback is provided to, or is available to be provided to, the input device.

In some embodiments, the reference location is located at a longitudinal position along the medical instrument that is coplanar with a hard stop location that limits deflection of the reference location.

In some embodiments, the medical instrument includes a force sensor unit and an end effector coupled to the distal end portion of the instrument. The force sensor unit includes a beam and one or more strain sensors coupled to the beam. The moment (e.g., the couple) is determined based on output of the strain sensor(s).

In some embodiments, the multiple operations optionally include any of the methods or operations disclosed herein. Additionally, the medical instrument optionally includes any of the structures or combinations of the structures disclosed herein.

These and other features, aspects, and advantages of the present invention will become better understood with reference to the following description and drawings.

### BRIEF DESCRIPTION OF THE DRAWINGS

FIGS. 1A and 1B are diagrammatic illustrations of a portion of a known medical device including a force sensor unit in a first configuration (FIG. 1A) and a second configuration (FIG. 1B).

FIGS. 2A and 2B are rigid body mechanics diagrams of the portion of the medical device shown in FIGS. 1A and 1B in the first configuration (FIG. 2A) and showing an exaggerated beam displacement (FIG. 2B).

FIG. 2C is a rigid body mechanics diagram of the portion of the medical device shown in FIGS. 1A and 1B being analyzed at a point of contact.

FIG. 3A is a graph showing the surface strain along the length of a beam of a force sensor unit when a single point of contact occurs.

FIG. 3B is a graph showing the determined force (Y-axis) as a function of the actual force (X-axis) to demonstrate determined force distortion.

FIG. 4 is a diagrammatic plan view of a minimally invasive teleoperated medical system according to an embodiment being used to perform a medical procedure, such as surgery.

FIG. 5 is a diagrammatic plan view of the minimally invasive teleoperated medical system of FIG. 4 being used to perform a medical procedure, such as surgery.

FIG. 6 is a front perspective view of a user control console of the minimally invasive teleoperated surgery system shown in FIG. 5, according to an embodiment.

FIG. 7 is a perspective view of an input device of the user console shown in FIG. 6.

FIG. 8 illustrates a displayed view of a surgical site as presented to an operator of the minimally invasive teleoperated surgery system by the user control console shown in FIG. 6.

FIG. 9 is a front perspective view of an optional auxiliary unit of the minimally invasive teleoperated surgery system shown in FIG. 5.

FIG. 10A is a side elevation view of a manipulator unit, including a plurality of manipulators and instruments, of the minimally invasive teleoperated surgery system shown in FIG. 5.

FIG. 10B is a diagrammatic illustration of a medical instrument supported by the manipulator unit shown in FIG. 10A.

FIG. 11A is a diagrammatic illustration of a portion of a medical instrument positioned within a cannula and including a force sensor unit.

FIG. 11B is a diagrammatic illustration of a portion of a medical instrument including a force sensor unit in a neutral orientation.

FIG. 12 is an enlarged view of a portion of the medical instrument of FIG. 11B indicated by the region K₁.

FIG. 13 is a diagrammatic illustration of a portion of a medical instrument of FIG. 11B including a force sensor unit in a deflected orientation.

FIG. 14 is an enlarged view of a portion of the medical instrument of FIG. 13 in the deflected orientation indicated by the region K₁ in FIG. 13.

FIG. 15 is a graph showing the determined force (Y-axis) as a function of the actual force (X-axis) during operation of the medical instrument of FIG. 11B on a condition in which the beam deflection is less than a deflection threshold and on a condition in which the beam deflection is larger than the deflection threshold.

FIG. 16 is a rigid body mechanics diagram of the medical instrument shown in FIG. 11B, showing an applied force at the end effector.

FIG. 17 is a rigid body mechanics diagram of the medical instrument shown in FIG. 11B, showing the applied force from FIG. 16 being resolved into an applied force at a reference location and a resulting moment.

FIG. 18 is a diagrammatic illustration of one configuration of the strain gauge resistors of the force sensor unit shown in FIGS. 11-14.

FIG. 19 is a diagrammatic illustration of one configuration of the force sensor unit shown in FIGS. 11-14, showing two half-bridge circuits formed with strain gauge resistors.

FIG. 20 is a perspective view of a medical device assembly according to an embodiment.

FIG. 21 is a side view of the medical device assembly of FIG. 20 showing selected instrument portions exposed, according to an embodiment.

FIG. 22 is a side view of one configuration of the force sensor unit shown in FIG. 21.

FIG. 23 is a is an electrical schematic illustration of one configuration of the force sensor unit shown in FIG. 22.

FIG. 24A is an enlarged illustration of a proximal portion of the force sensor unit shown in FIG. 22 indicated by the region K₂ in FIG. 22.

FIG. 24B is an enlarged illustration of a distal portion of the force sensor unit shown in FIG. 22 indicated by the region K₃ in FIG. 22.

FIG. 25 is an enlarged illustration of a portion of the force sensor unit shown in FIG. 22 illustrating an alternative arrangement of the strain gauge resistors.

FIG. 26 is an enlarged illustration of a portion of the force sensor unit shown in FIG. 22 illustrating an alternative arrangement of the strain gauge resistors.

FIG. 27 is a diagrammatic illustration of a controller for use with a minimally invasive teleoperated surgery system according to an embodiment.

FIG. 28 is a flow chart of a method of control for a surgical system according to an embodiment.

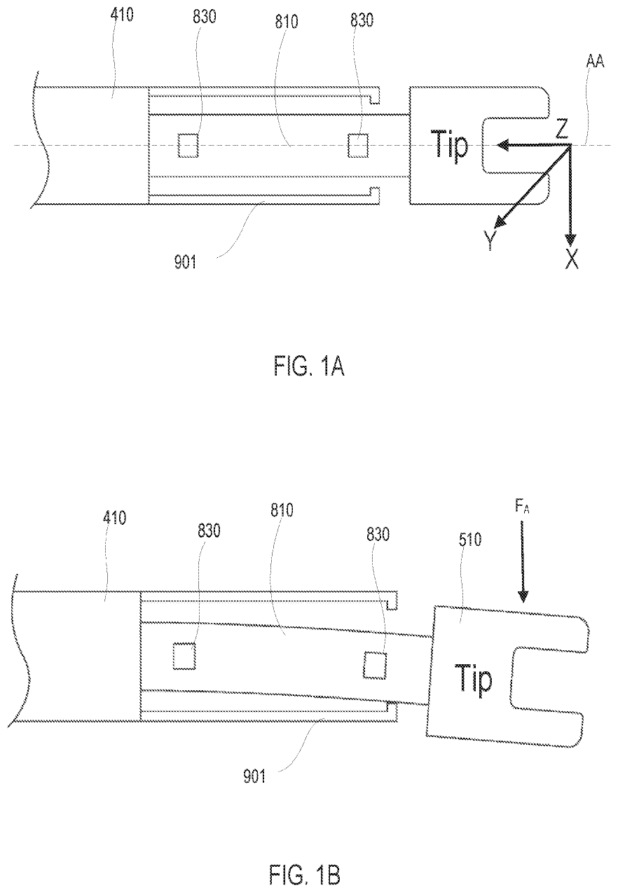

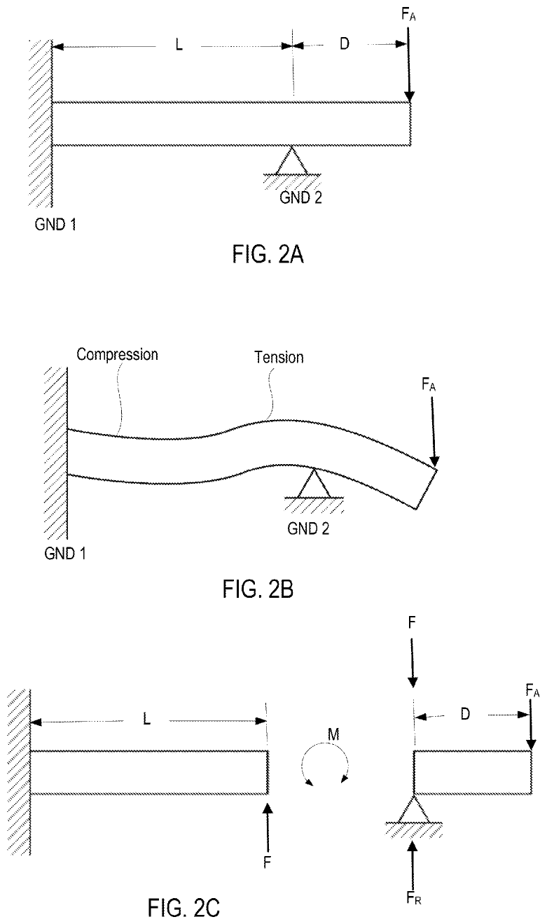

### DETAILED DESCRIPTION

Reference now will be made in detail to embodiments of the invention, one or more examples of which are illustrated in the drawings. Each example is provided by way of explanation of the invention, not limitation of the invention. In fact, it will be apparent to those skilled in the art that various modifications and variations can be made in the present invention without departing from the scope or spirit of the invention. For instance, features illustrated or described as part of one embodiment can be used with another embodiment to yield a still further embodiment. Thus, it is intended that the present invention covers such modifications and variations as come within the scope of the appended claims and their equivalents.

The embodiments described herein can advantageously be used in a wide variety of operations associated with minimally invasive surgery, including grasping, cutting, and otherwise manipulating tissue. The medical instruments or devices of the present application enable motion in three or more degrees of freedom (DOFs). For example, in some embodiments, an end effector of the medical instrument can move with reference to the main body of the instrument in three mechanical DOFs, e.g., pitch, yaw, and roll (shaft roll). There may also be one or more mechanical DOFs in the end effector itself, e.g., two jaws, each rotating with reference to a clevis (2 DOFs), and in turn the clevis may optionally rotate with reference to a more proximal clevis (one DOF) or other mechanical reference. Thus, in some embodiments, the medical instruments or devices of the present application may enable motion in six or more DOFs, including all six Cartesian DOFs. Further, the embodiments described herein are used to deliver a modified force feedback to a system operator in response to forces exerted on (or by) a distal end portion of the instrument during use under certain operating conditions.

Persons of skill in the art will understand that during surgery, various forces will be applied to a surgical instrument's distal end. In some situations the applied force may directly act on the instrument, in some situations the applied force may be a reactive force as a result of the instrument acting on another object, and in some situations, the applied force may be a combination of a direct action force and a reactive force. The weight of retracted tissue and another instrument striking the instrument are examples of forces directly acting on the instrument. Alternatively, the force exerted by resilient or hard tissue or when tightening suture are examples of reactive forces acting on the instrument as it moves against these objects. In this description, any one of the directly acting, reactive, or combined direct and reactive forces are referred to as an applied force on the instrument. When using a hand-operated instrument, the clinician experiences this applied force as a direct haptic sensation through the instrument. But when using a motor-powered instrument, this applied force is isolated from the human clinical operator, and so the applied force is detected, measured, and fed back to the clinical operator via a haptic force feedback system.

Generally, the present disclosure is directed to systems and methods for controlling a surgical system such as a minimally invasive teleoperated surgery system. In particular, the present disclosure may include a system and methods that may facilitate the modification of the haptic feedback delivered to the operator of the surgical system in relation to a restricted feedback condition of the surgical system. The restricted feedback condition may correspond to a condition of the surgical system wherein the haptic feedback generated based on the determined force may not accurately reflect the forces acting on the instrument. For example, the restricted feedback condition may correspond to a portion of an operating range of the medical device in which the force measured by the surgical system deviates from the actual force exerted on (or by) the distal end of the medical device.

The restricted feedback condition can, for example, correspond to an operating condition of the surgical system in which a deflection of one structure of the instrument places a portion of the instrument in contact with another structure of the instrument (e.g., a hard stop location that limits further deflection of the structure). In such an operating condition, the limiting structure exerts a reactive force on the deflecting portion of the instrument. The reactive force is in opposition to the applied force that generated the deflection. Accordingly, indications of force received from the instrument may not accurately reflect the applied force. Therefore, it is desirable to detect when the deflecting portion of the instrument is in contact with the deflection-limiting structure because measurements of the applied force may not correspond to the actual magnitude of the applied force. As described herein, the magnitude of a moment (and force) of an end effector of the instrument provides an indication of the contact between the portion of the instrument and the more rigid structure. The systems and methods disclosed herein facilitate the detection of such an operating condition and the generation of a corresponding modification of the haptic feedback delivered to the operator of the surgical system.

In order to determine the magnitude of the moment (and force), and thus the deflection of the instrument, a measured output voltages (e.g., a voltage differential) is received from a first half-bridge circuit and a measured output voltage (e.g., a voltage differential) is received from a second half-bridge circuit. This pair of output voltages can be used with Eqs. 12 and 13 to solve the two equations for the two unknowns, a measured force and a pure moment (e.g., a couple) at a reference location. Based on the resultant values of the measured force and the pure moment, the deflection can be determined utilizing Eq. 5. Since clearances between the various portions of the medical instrument are known, a deflection determined using Eq. 5 that is greater than the clearance is indicative of contact between a portion of the instrument and another structure of the instrument (e.g., a hard stop location that limits further deflection of the structure).

As disclosed herein, when the position, condition, and/or operation of the medical device of the surgical system is in the restricted feedback condition, the force feedback (e.g., haptic, visual, or auditory feedback) delivered to the operator of the surgical system may be reduced/limited relative to a designed haptic feedback. The reduction/limiting (e.g., disabling) of the haptic feedback facilitates continued, accurate control of the surgical system by the operator under conditions in which the feedback may otherwise be inaccurate and/or unreliable.

In addition to affecting the haptic feedback provided to the operator of the surgical system, the systems and methods disclosed herein may also pause (e.g., hold in place) an operation of the surgical system when at or approaching a transition between a restricted feedback condition and an unrestricted feedback condition (e.g., a designed feedback type, magnitude, and/or direction for a given condition of the instrument). In one optional embodiment, an indication of the transition is presented to the operator. Upon the operator's acknowledgment of the indication, the operation of the surgical system is resumed and the appropriate haptic feedback is provided to the operator. For example, when transitioning from the unrestricted feedback condition to the restricted feedback condition, upon acknowledgment, the haptic feedback delivered to the operator may be reduced or disabled. Similarly, when transitioning from the restricted feedback condition to the unrestricted feedback condition, upon acknowledgment, designed haptic feedback may be delivered to the operator. It should be appreciated that the pausing of the operation of the surgical system until the acknowledgment of the modification of the haptic feedback is received may facilitate transitions between feedback conditions and therefore the accurate control of the surgical system.

As used herein, the term “about” when used in connection with a referenced numeric indication means the referenced numeric indication plus or minus up to 10 percent of that referenced numeric indication. For example, the language “about 50” covers the range of 45 to 55. Similarly, the language “about 5” covers the range of 4.5 to 5.5.

As used in this specification and the appended claims, the word “distal” refers to direction towards a work site, and the word “proximal” refers to a direction away from the work site. Thus, for example, the end of a tool that is closest to the target tissue is the distal end of the tool, and the end opposite the distal end (i.e., the end manipulated by the user or coupled to the actuation shaft) is the proximal end of the tool.

Further, specific words chosen to describe one or more embodiments and optional elements or features are not intended to limit the invention. For example, spatially relative terms-such as “beneath”, “below”, “lower”, “above”, “upper”, “proximal”, “distal”, and the like—may be used to describe the relationship of one element or feature to another element or feature as illustrated in the figures. These spatially relative terms are intended to encompass different positions (i.e., translational placements) and orientations (i.e., rotational placements) of a device in use or operation in addition to the position and orientation shown in the figures. For example, if a device in the figures is turned over, elements described as “below” or “beneath” other elements or features would then be “above” or “over” the other elements or features. Thus, the term “below” can encompass both positions and orientations of above and below. A device may be otherwise oriented (e.g., rotated 90 degrees or at other orientations) and the spatially relative descriptors used herein interpreted accordingly. Likewise, descriptions of movement along (translation) and around (rotation) various axes includes various spatial device positions and orientations. The combination of a body's position and orientation define the body's pose.

Similarly, geometric terms, such as “parallel”, “perpendicular”, “round”, or “square”, are not intended to require absolute mathematical precision, unless the context indicates otherwise. Instead, such geometric terms allow for variations due to manufacturing or equivalent functions. For example, if an element is described as “round” or “generally round,” a component that is not precisely circular (e.g., one that is slightly oblong or is a many-sided polygon) is still encompassed by this description.

In addition, the singular forms “a”, “an”, and “the” are intended to include the plural forms as well, unless the context indicates otherwise. The terms “comprises”, “includes”, “has”, and the like specify the presence of stated features, steps, operations, elements, components, etc. but do not preclude the presence or addition of one or more other features, steps, operations, elements, components, or groups.

Aspects of the invention are described using a da Vinci® surgical system (commercialized by Intuitive Surgical, Sunnyvale, California) as an example surgical system form. Knowledgeable persons will understand that inventive aspects disclosed herein may be embodied and implemented in various ways, including computer-assisted, non-computer-assisted, and hybrid combinations of manual and computer-assisted embodiments and implementations. Implementations on da Vinci® Surgical Systems are not to be considered as limiting the scope of the inventive aspects disclosed herein. As applicable, inventive aspects may be embodied and implemented in both relatively smaller, hand-held, hand-operated devices and relatively larger systems that have additional mechanical support.

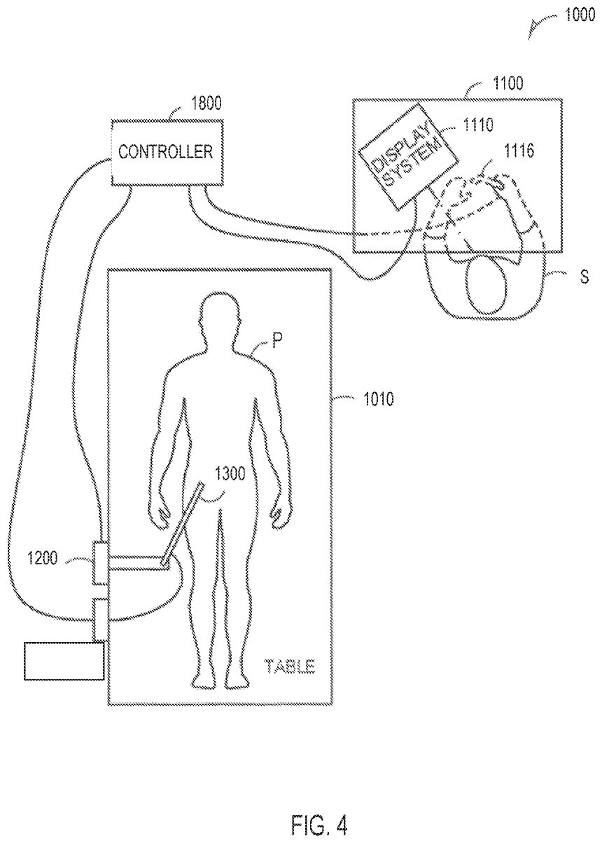

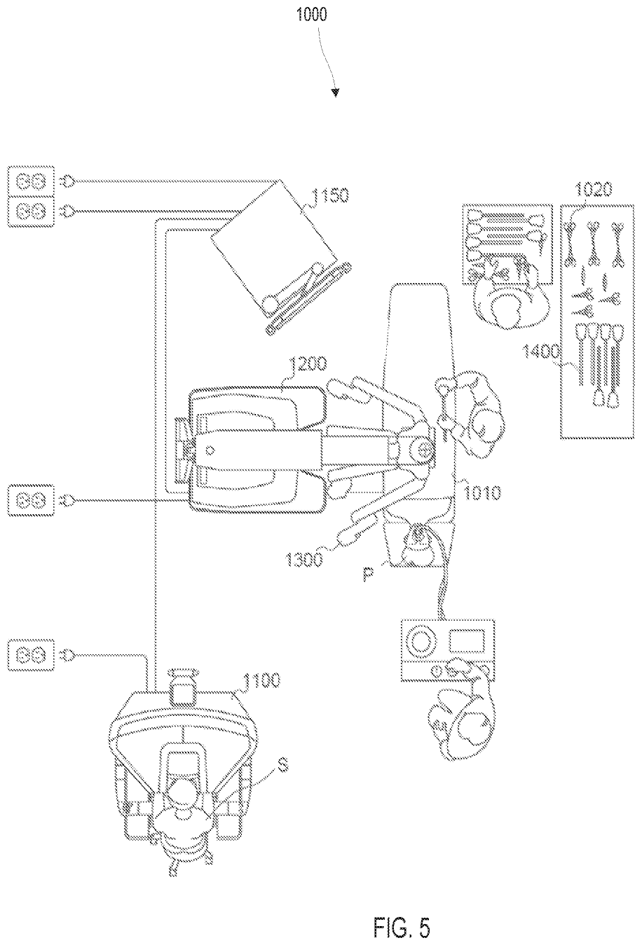

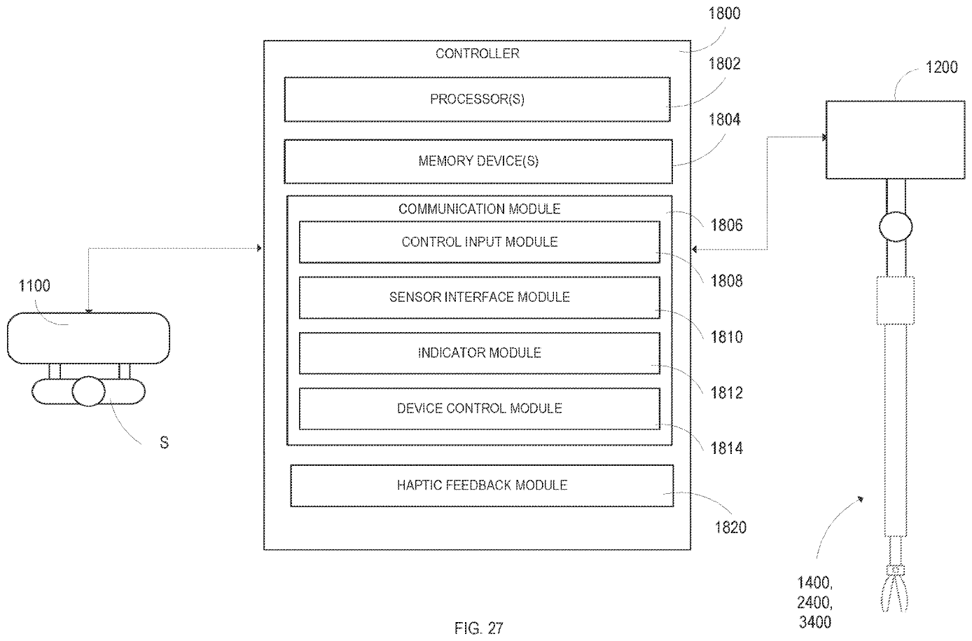

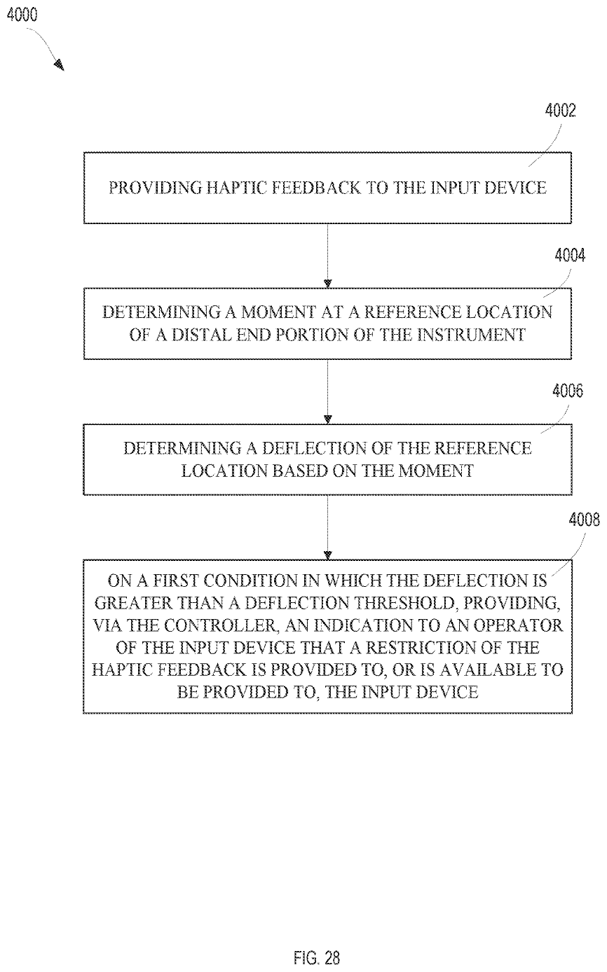

FIGS. 4 and 5 are plan view illustrations of a teleoperated surgical system 1000 that operates with at least partial computer assistance (a “telesurgical system”). Both the telesurgical system 1000 and its components are considered medical devices. The telesurgical system 1000 is a Minimally Invasive Robotic Surgical (MIRS) system used for performing a minimally invasive diagnostic or surgical procedure on a Patient (P) who is lying on an Operating table 1010. The system is made of various optional components, such as a user control unit 1100 for use by a surgeon or other skilled clinician (S) (e.g., operator of the surgical system) during the procedure. The telesurgical system 1000 further includes a manipulator unit 1200 (popularly referred to as a surgical robot) and an optional auxiliary equipment unit 1150. The manipulator unit 1200 includes an arm assembly 1300 and an instrument (e.g., a surgical instrument tool assembly) optionally removably coupled to the arm assembly. The manipulator unit 1200 can manipulate at least one removably coupled instrument 1400 through a minimally invasive incision in the body or natural orifice of the patient (P) while the surgeon (S) views the surgical site and controls movement of the instrument 1400 through control unit 1100 with the assistance of a controller 1800. Further details of the controller 1800 are described below with reference to FIG. 27. An image of the surgical site is obtained by an endoscope, such as a stereoscopic endoscope, which can be manipulated by the manipulator unit 1200 to orient the endoscope. The auxiliary equipment unit 1150 can be used to process the images of the surgical site for subsequent display to the surgeon (S) via a display system 1110 of the user control unit 1100. The number of instruments 1400 used at one time will generally depend on the diagnostic or surgical procedure and the space constraints within the operating room, among other factors. If it is necessary to change one or more of the instruments 1400 being used during a procedure, an assistant removes the instrument 1400 from the manipulator unit 1200 and replaces it with another instrument 1400 from a tray 1020 in the operating room. Although shown as being used with the instruments 1400, any of the instruments described herein can be used with the telesurgical system 1000. It should be appreciated that the surgical site is either at the skin surface or within at least a portion of the body of the patient (P).

The user control unit 1100 is shown in FIGS. 4 and 5 as being in the same room as the patient (P) so that the surgeon (S) can directly monitor the procedure, be physically present if necessary, and speak to an assistant directly rather than over the telephone or other communication medium. In other embodiments, however, the user control unit 1100 and the surgeon (S) can be in a different room, a completely different building, or other location remote from the patient (P), allowing for remote surgical procedures.

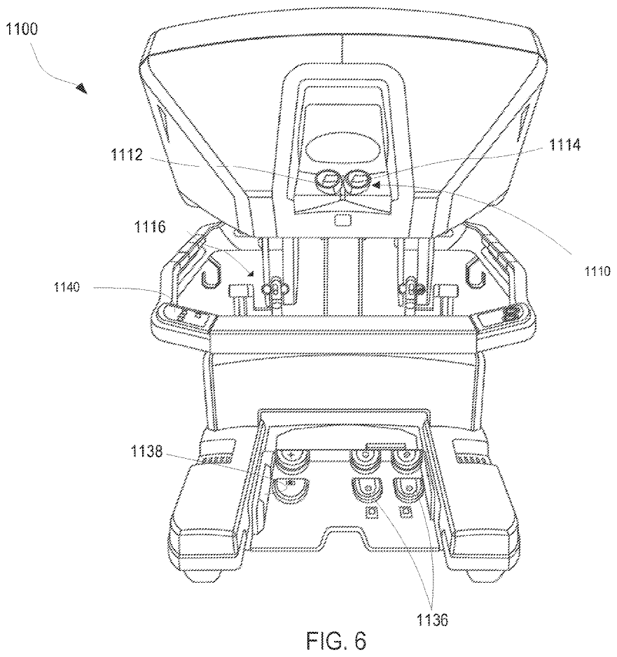

FIG. 6 is a perspective view of the control unit 1100. The user control unit 1100 includes one or more input control devices 1116 configured to be held by the surgeon (S), which in turn cause the manipulator unit 1200 to manipulate one or more instrument (e.g., tools, medical devices, and/or surgical instruments). The input control devices 1116 provide at least the same degrees of freedom as instruments 1400 with which they are associated to provide the surgeon (S) with telepresence, or the perception that the input control devices 1116 are integral with the instruments 1400. In this manner, the user control unit 1100 provides the surgeon (S) with a strong sense of directly controlling the instruments 1400. To this end, impressions (e.g., haptic feedback) of position, force, strain, or tactile feedback sensors (not shown) or any combination of such sensations, are delivered from the instruments 1400 back to the surgeon (S) through the one or more input control devices 1116.

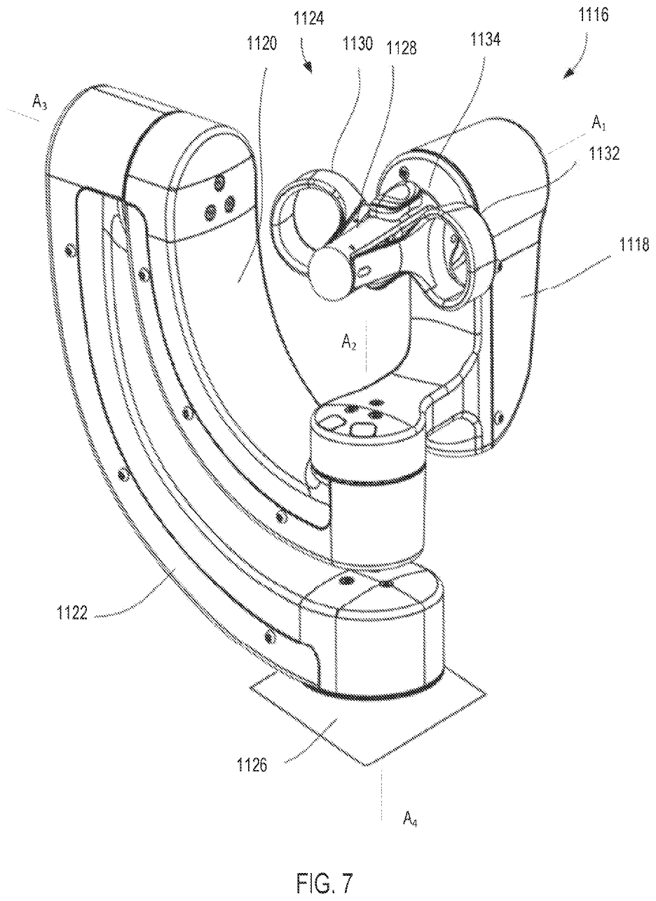

FIG. 7 is a perspective view of an input control device 1116 configured to be held with at least a portion of surgeon's (S) hand, according to an embodiment. In such a configuration, links are interconnected in a gimbal arrangement so that the input control device 1116 includes a first link 1118 (which functions as a first gimbal link), a second link 1120 (which functions as a second gimbal link), a third link 1122 (which functions as a third gimbal link), and an input handle 1124. The input control device 1116 is mounted to a base portion 1126, which is a distal portion of an kinematic arm that itself is a part of a user control unit, such as the user control unit 1100 described herein. Although an individual mechanically grounded hand input control device is shown for illustrative purposes, other optional gimbal and non-gimbal configurations, and mechanically grounded and ungrounded configurations, are known and may be used in accordance with the inventive aspects described herein.

As shown, the input handle 1124 includes a handle portion 1128, an optional first grip lever input 1130, an optional second grip lever input 1132, and a handle input shaft 1134. In an embodiment, the handle input shaft 1134's long axis defines a first rotational axis A₁ (which in this description functions as a roll axis; the term roll is arbitrary) and is rotatably coupled to the first link 1118. The handle portion 1128 is supported on the handle input shaft 1134 and is configured to be rotated relative to the first link 1118 about the first rotational axis A₁. The input shaft 1134 extends at least partially within the first link 1118. The first handle input 1130 and the second handle input 1132 can be manipulated to produce a desired action at the instrument end effector (not shown) operatively coupled to the input device 1116 and its handle 1124. For example, in some embodiments, the first grip lever input 1130 and the second grip lever input 1132 can be squeezed together to produce a gripping movement at the end effector. The first and second grip lever inputs 1130, 1132 are similar to the grip members shown and described in U.S. Patent Application Pub. No. US 2020/0015917 A₁ (filed Jun. 14, 2019), entitled “Actuated Grips for Controller,” which is incorporated herein by reference in its entirety for all purposes. In other embodiments, however, the input handle 1124 need not include the grip lever inputs, or the grip lever inputs are illustrative of other optional hand-operated control inputs (e.g., buttons, levers, switches, wheels) that may be used in other configurations.

As depicted in FIG. 6, in an embodiment, at least one of the user control unit 1100 may be configured to be engaged via a portion of at least one foot of the surgeon (S). In such a configuration, the user control unit 1100 can include at least one pedal assembly 1136 and/or at least one foot-activated switch assembly 1138. Each pedal assembly 1136 and/or foot-activated switch assembly 1138 may include at least one switch (not shown) activated by the respective assembly. The surgical system 1000 may detect that one or more electrosurgical tools are mounted to the manipulator unit 1200 and may assign the appropriate control functions to the pedal assembly 1136 and/or foot-activated switch assembly 1138.

In some embodiments, the user control unit 1100 includes one or more optional touchpads 1140 configured to receive an input from the surgeon (S). The touchpad(s) 1140 may, for example, be a liquid crystal display (LCD) screen. The touchpad(s) 1140 may, as depicted in FIG. 6, be mounted in an arm rest or at another suitable location of the user control unit 1100. The surgeon (S) may utilize the touchpad(s) 1140 to access various operations, protocols, and/or settings of the surgical system 1000, such as user accounts, ergonomic settings, preferences, equipment configurations, operational status commands, and/or other similar processes as described herein. Additionally, the human clinical operator may use the touchpad(s) 1140 to acknowledge various system messages, alerts, and/or warnings as described herein.

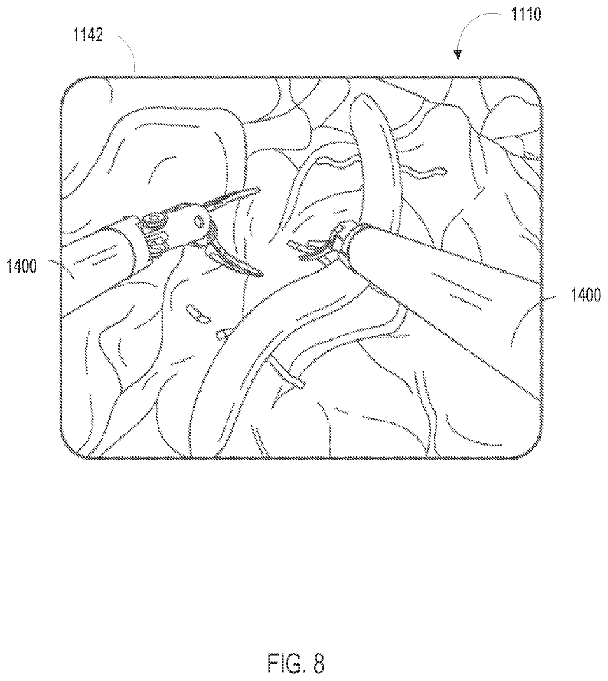

As further depicted in FIG. 6, the user control unit 1100 includes a display system 1110. In other user control unit examples, the display is separate from the console structure and may be, for example, mounted on a wall or other support structure. As depicted in FIG. 8, the display system 1110 defines a field-of-view 1142 of the operator (S). In some embodiments, the display system 1110 is stereoscopic and includes a left eye display 1112 and a right eye display 1114 for presenting the surgeon (S) with a coordinated stereoscopic view of the surgical site that enables depth perception. In other embodiments a monoscopic display may be used. Various other stereoscopic and monoscopic display systems are known and are contemplated to be within the scope of various inventive aspects described herein. True three-dimensional displays are contemplated. Such stereoscopic and monoscopic display systems may be mechanically grounded as shown, or they may be mechanically ungrounded and embodied in devices such as head mounted displays. Although not shown in FIG. 8, it is well understood that display system 1110 may optionally display various messages to the operator that include information aspects as described herein (e.g., information about the status of a haptic feedback system).

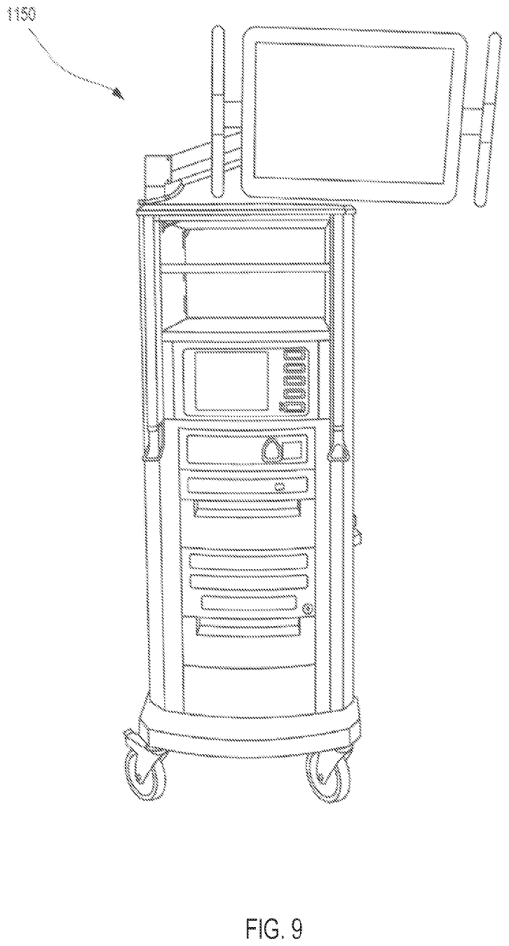

FIG. 9 is a perspective view of the auxiliary equipment unit 1150. In some embodiments the auxiliary equipment unit 1150 is coupled with the endoscope and includes one or more processors to process captured images for subsequent display, such as via the display system 1110 of the user control unit 1100, or on another suitable display located locally (e.g., on the unit 1150 itself as shown, on a wall-mounted display) and/or remotely. For example, if a stereoscopic endoscope is used, the auxiliary equipment unit 1150 processes the captured images to present the surgeon (S) with coordinated stereo images of the surgical site via the left eye display 1112 and the right eye display 1114. Such coordination optionally includes alignment between the opposing images and optionally includes adjusting the stereo working distance of the stereoscopic endoscope. As another example, image processing can include the use of previously determined camera calibration parameters to compensate for imaging errors of the image capture device, such as optical aberrations.

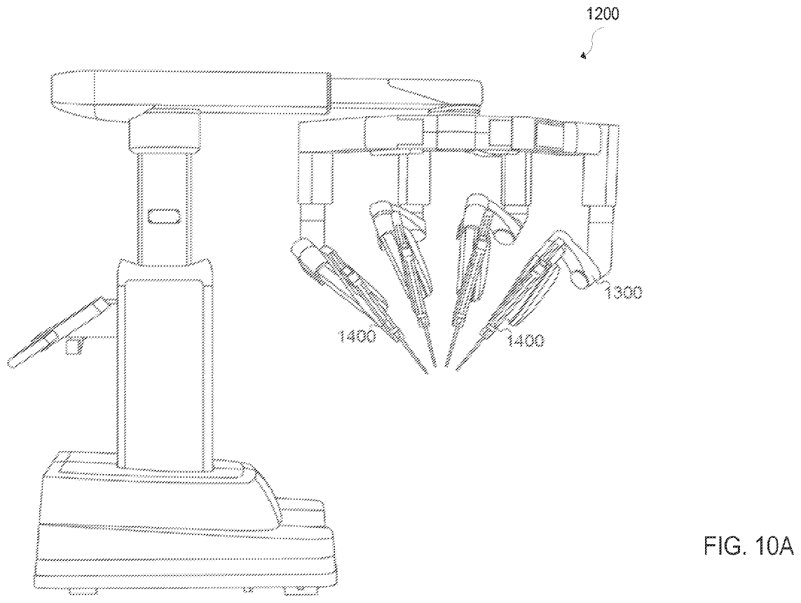

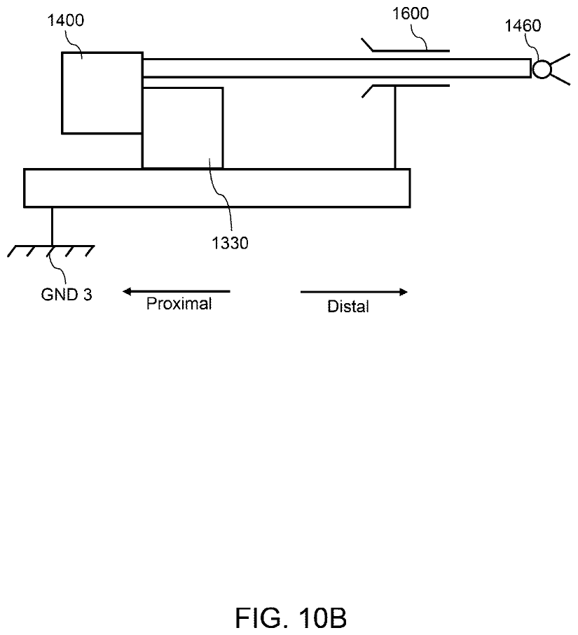

FIG. 10A is a side view of the manipulator unit 1200. The manipulator unit 1200 includes the components (e.g., arms, linkages, motors, sensors, and the like) to provide for the manipulation of the instruments 1400 and an imaging device (e.g., the endoscope) used for the capture of images of the site of the procedure. Specifically, the instruments 1400 and the imaging device can be manipulated by teleoperated mechanisms having one or more mechanical joints. Moreover, the instruments 1400 and the imaging device are positioned and manipulated through incisions or natural orifices in the patient (P) in a manner such that a center of motion remote from the manipulator and typically located at a position along the instrument shaft is maintained at the incision or orifice by either kinematic mechanical or software constraints. In this manner, the incision size can be minimized and tissue damage minimized at the incision.

FIG. 10B is a diagrammatic illustration of the instrument 1400 and a cannula structure 1600 supported by the arm assembly 1300. As depicted, the arm assembly 1300 includes an instrument carriage 1330. The instrument carriage 1330 includes teleoperated actuators (not shown) to provide controller motions to the instrument 1400, which translates into a variety of movements of a tool or tools at an end effector 1460 of the instrument 1400. The instrument carriage 1330 is also translatable relative to the arm assembly 1300, for example, along an insertion axis extending between a proximal end and a distal end of the arm assembly 1300 for insertion and removal of the instrument into a patient. The translation of the instrument carriage 1330 develops a corresponding linear motion, relative to a longitudinal axis (e.g., in a distal or proximal direction) of the end effector 1460. As depicted, the arm assembly 1300 has a mechanical ground connection GND 3 with the manipulator unit 1200. In some embodiments, the cannula structure 1600 is removably coupled to the arm assembly 1300. In other words, the cannula structure 1600 can be rigidly coupled to the arm assembly 1300. The cannula 1600 is configured to surround at least a portion of the instrument 1400 to facilitate access of the surgical site by the end effector 1460.

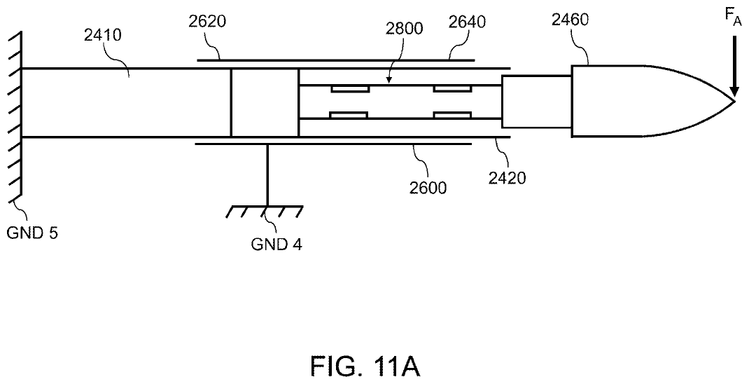

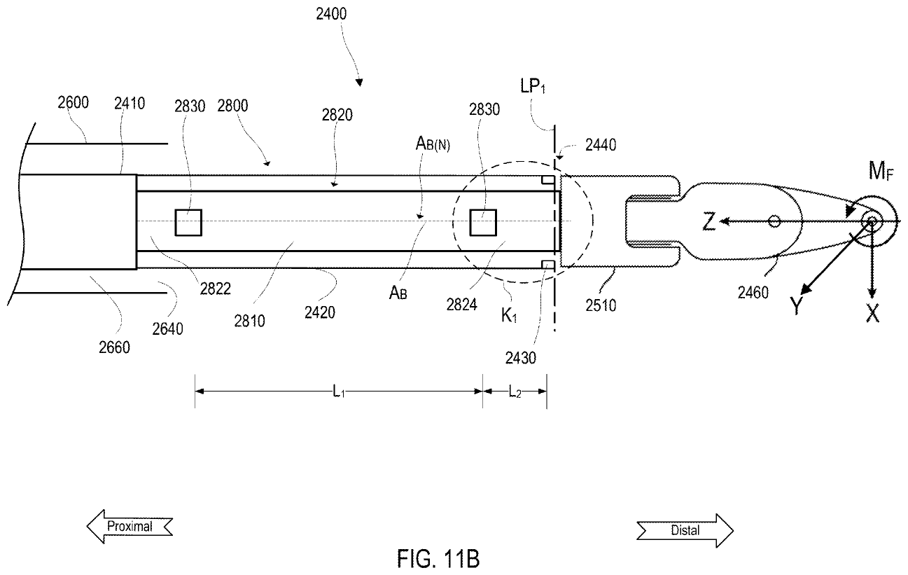

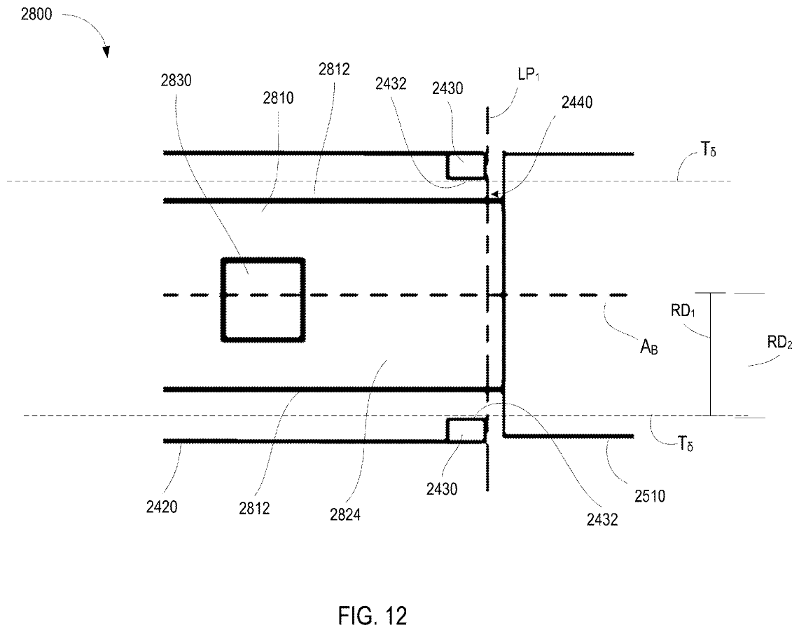

FIG. 11A is a diagrammatic illustration of a portion of an instrument 2400 positioned within a cannula 2600 according to an embodiment. FIG. 11B is a schematic illustration of a distal end portion of a surgical instrument 2400, according to an embodiment, and FIG. 12 is an enlarged view of a portion of the surgical instrument 2400 of FIG. 11B indicated by the region K₁. As depicted, instrument 2400 extends through the cannula structure 2600, and a portion of the surgical instrument 2400 is surrounded by cannula 2600. The cannula structure 2600 has a proximal end 2620 and a distal end 2640. The cannula structure 2600 has a central channel 2660 that extends between the proximal end 2620 and the distal end 2640, through which the surgical instrument 2400 is inserted during a medical procedure. The cannula structure 2600 may be a straight cannula as shown. In additional embodiments, the cannula structure 2600 may optional be a curved cannula having a combination of linear and nonlinear sections, a cannula with multiple non-parallel linear sections, a cannula with multiple curve sections having different characteristics, and/or a cannula with other combinations of linear and nonlinear sections. The cannula structure 2600 has a mechanical ground connection GND 4 with an arm assembly (e.g., arm assembly 1300) of the surgical system.

The surgical instrument 2400 includes a shaft 2410 and a force sensor unit 2800 (e.g., a force sensor assembly), which includes a resiliently deflectable beam 2810 and one or more strain sensors 2830 mounted on a surface along the beam 2810 to sense strain that results from beam 2810 deflecting. The shaft 2410 includes a distal end portion, and a proximal end portion 2822 of the beam 2810 is coupled to the distal end portion of the shaft. In some embodiments, proximal end portion of the beam is directly coupled to the distal end portion of the shaft 2410, and in other embodiments the proximal end portion 2822 of the beam is coupled to the proximal end portion 2822 of the beam via another coupling component (such as a mechanical anchor or coupler, not shown). In some embodiments a proximal end portion of the shaft 2410 is coupled to a mechanical structure (not shown) configured to move one or more components of the surgical instrument, such as, for example, the end effector 2460. In other words, in some embodiments, the shaft 2410 as a mechanical ground connection GND 5 with the arm assembly (e.g., via an instrument carriage) of the surgical system. Thus, the beam 2810 couples the connecting link 2510 (and the end effector 2460) to the shaft 2410 in a cantilevered configuration anchored at the proximal end portion 2822 of the beam.

One or more distal end components of the instrument (e.g., a surgical end effector, a wrist assembly, and the like) are connected to beam 2810's distal end portion 2824 via connecting link 2510. As shown, an example end effector 2460 may by coupled at a distal end portion 2824 of the beam 2810 (i.e., at a distal end portion of the surgical instrument 2400). The end effector 2460 can include, for example, articulatable jaws, a cautery instrument, and/or any other suitable surgical tool that is coupled to a link 2510 (e.g., a proximal clevis pin). In some embodiments, the link 2510 can be included within a wrist assembly having multiple articulating links. In some embodiments the link 2510 is included as part of the end effector 2460.

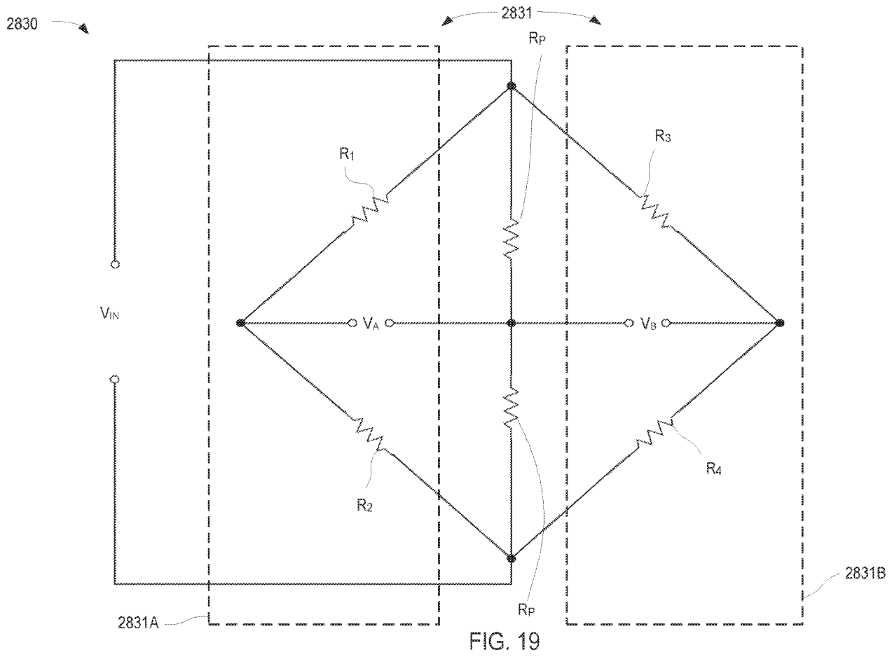

The one or more strain sensors 2830 are optionally made of one or more electrical strain sensing circuits (e.g., bridge circuits 2831—see FIGS. 18 and 19), and other strain sensor configurations are contemplated (e.g., optical fiber Bragg grating sensors, piezoelectric sensors, and the like). As described herein, each bridge circuit 2831 (and also each strain sensor) includes one or more strain gauges (e.g., tension strain gauge resistor(s) or compression strain gauge resistor(s)). It should be appreciated that the beam 2810 can include any number of strain sensors 2830 in various arrangements.

In some embodiments, a shroud 2420 optionally surrounds at least a portion of the beam 2810.

As shown, the beam 2810 of the force sensor unit 2800 includes a middle portion 2820 that is between a proximal end portion 2822 and a distal end portion 2824. The beam 2810 center axis (A_{B}) is defined by end portions 2822, 2824 and centered on the beam. In some embodiments center axis A_{B} is aligned (collinear) with a similar center axis (not shown) of the instrument shaft 2410, and in other embodiments these two axes are not collinear. As described below, the deflection of the beam from axis A_{B} in the beam's undeflected state (as measured by the strain sensors 2830) can be correlated to applied forces applied to the end effector 2460.

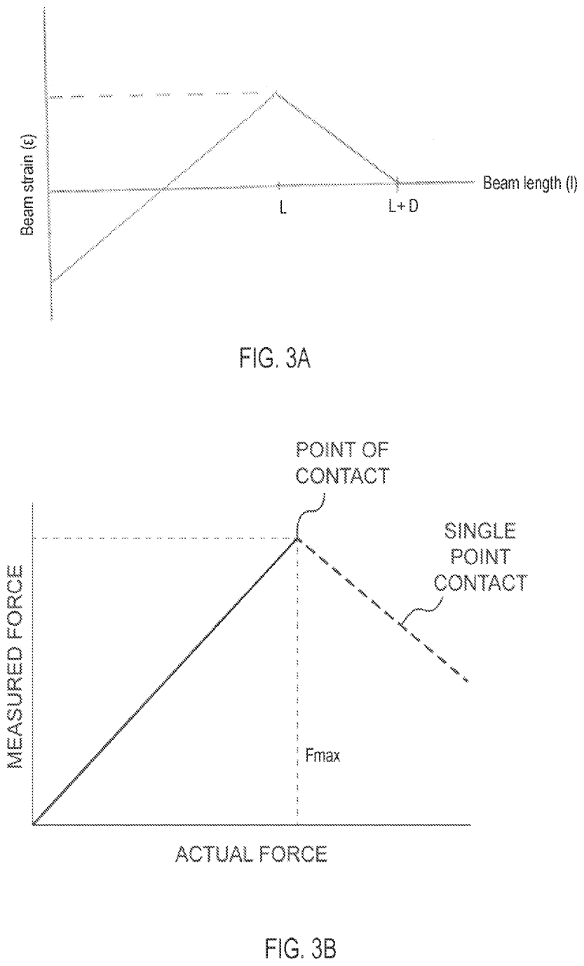

Generally, Cartesian X, Y, and Z direction forces (direct or reactive) are imparted on the end effector 2460. In practice, an applied force can be deconstructed into its Cartesian components. Resolved moments (MF)corresponding to these X, Y, or Z forces are likewise imparted to the end effector, and the magnitude of such moments depends on a defined origin for a moment. For example, as shown in FIG. 3 an applied force F_{A} in the X direction results in a resolved moment (MF) about the Y-direction axis. More specifically, as described herein applied forces acting generally normal to the beam center axis A_{B} (i.e., the X- and Y-direction forces as shown) will result in a corresponding moment on beam 2810. Forces acting in the Z-direction along the center axis A_{B} are a special case because they act through center axis A_{B}, and so through the defined origin with a resulting moment of zero magnitude.

Referring to FIGS. 18 and 19, the strain gauge resistors R₁, R₂, R₃, and R₄, which are included within strain sensors and which can be arranged into one or more bridge circuits 2831 (e.g., a Wheatstone bridge), can measure strain in the beam 2810 that can be used to determine the forces imparted on the end effector 2460 in the X and Y axis directions normal to the beam. These X and Y axes forces are transverse (e.g., perpendicular) to the Z axis (which is parallel or collinear with the longitudinal center axis (A_{B}) of the beam 2810). Such transverse forces acting upon the end effector 2460 cause a deflection (e.g., bending) of the beam 2810 (about either or both of the X axis or the Y axis), which because of its cantilever configuration results in a tensile strain imparted to one side of the beam 2810 and a compression strain imparted to the opposite side of the beam 2810. The strain gauge resistors R₁, R₂, R₃, and R₄ on the beam 2810 are used to determine such tensile and compression strains.

It will be understood that the exact location of an applied force (or force component) on the instrument is not determined, because the location along the length of the instrument at which the force (or force component) is applied is unknown. For example, a force applied directly to the distal tip of the end effector will cause the same sensed strain as a slightly larger force applied a short distance proximal of the end effector's distal tip due to an infinite number of force and moment pairs that would result in the same strain. This situation is also true for hand-operated instruments. In practice, this determined force effectively mimics a similar condition in a hand-held instrument and can be effectively used in a haptic force feedback system for a human clinical operator because of the relatively small differences in applied force locations at the instrument's distal end portion.

Therefore, the outputs of the strain gauge resistors R₁, R₂, R₃, and R₄ on the beam 2810 is correlated to a determined force (F). Moreover, as described herein, depending on the arrangement of the strain gauge resistors R₁, R₂, R₃, and R₄ (i.e., in two half-bridge circuits 2831), the strain gauge resistors R₁, R₂, R₃, and R₄ can be used to determine the moment (M) that results from an applied force on the instrument. It should be appreciated that the output of the force sensor unit 2800 may be used by a controller, such as the controller 1800 of system 1000 described above, to determine the haptic feedback to deliver to the surgeon (S) via the input control device(s) 1116.

Although shown as including only the force sensor unit 2800, in some embodiments, the instrument 2400 (or any of the instruments described herein) optionally include additional force sensor units to measure the axial force(s) (i.e., in the direction of the Z-axis parallel to the beam center axis (A_{B})) imparted on the end effector 2460. An axial force sensor unit in an example surgical instrument can comprise a deflectable planar diaphragm sensor that deflects in response to a force. Alternatively, a deflectable ferrite core can be used within an inductive coil may be used or a or a fiber Bragg grating formed within an optical fiber can be used, for example. Other axial force sensor units may be used to sense a resilient axial displacement of the shaft 2410 (e.g., relative to the proximally mounted mechanical structure, not shown). An axial force Fz imparted to the end effector 2460 can cause axial displacement of the shaft 2410 in a direction along a center axis of the shaft (substantially parallel to the beam center axis (A_{B})). The axial force Fz may be in the proximal direction (e.g., a reactive force resulting from pushing against tissue with the end effector) or it may be in the distal direction (e.g., a reactive force resulting from pulling tissue grasped with the end effector).

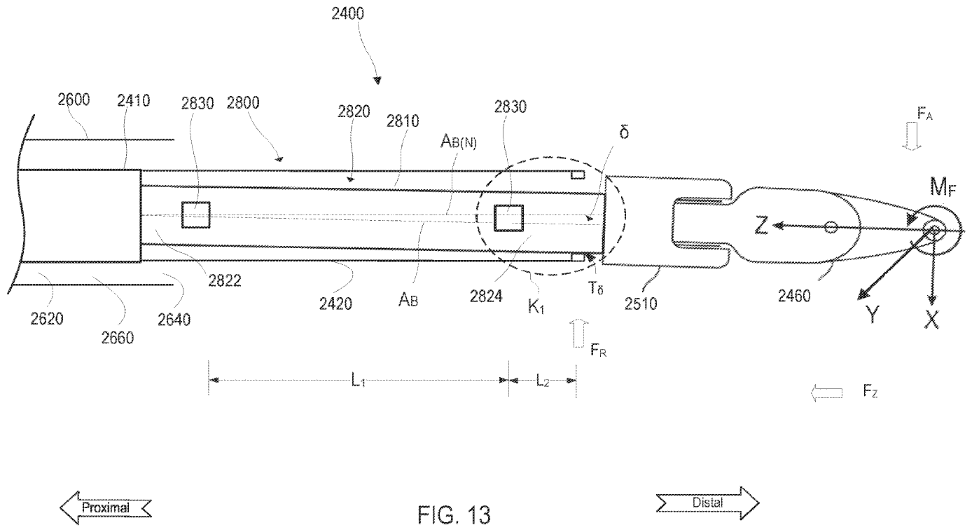

In some conditions, X and/or Y forces imparted on the end effector 2460, such as depicted in FIG. 13, may result in strain in the beam 2810 when the beam 2810 is deflected (6) (e.g., resiliently displaced). In other words, the X and/or Y forces deflect the center axis (A_{B}) of the beam 2810 away from a zero-force designed position (A_{B(N)}) of the center axis (A_{B}), and thus deflect center axis A_{B} relative to the longitudinal center axis of the shaft 2410. Said another way, the distal end portion 2824 of the beam 2810 can bend relative to a proximal end portion 2822 of the beam 2810 such that the distal end portion 2824 of the beam 2810 is displaced a deflection distance relative to the designed position (A_{B(N)}) of the center axis (A_{B}).

In some embodiments, beam deflection is limited by a hard stop location. For example, the shroud 2420 and/or the cannula structure 2600 can limit the displacement of the beam 2810. Such deflection limiting produces a reactive force (F_{R}) on the beam 2810. Thus, in some embodiments the shroud 2420 functions as a deflection limiting hard stop 2430. Similarly, in some embodiments, the cannula 2600 functions as a deflection limiting hard stop 2430. In some embodiments, the instrument can include one or more hard stop structures that function as a deflection limiting hard stop 2430. It should be appreciated that the hard stop 2430 can be a known point of contact between structures of the instrument and/or a structure coupled to the instrument 2400 at a specified longitudinal location to limit deflection of the beam 2810.

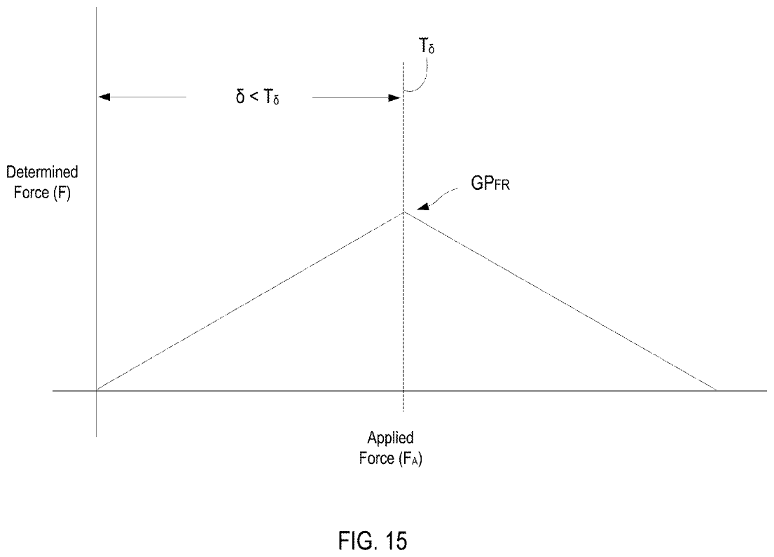

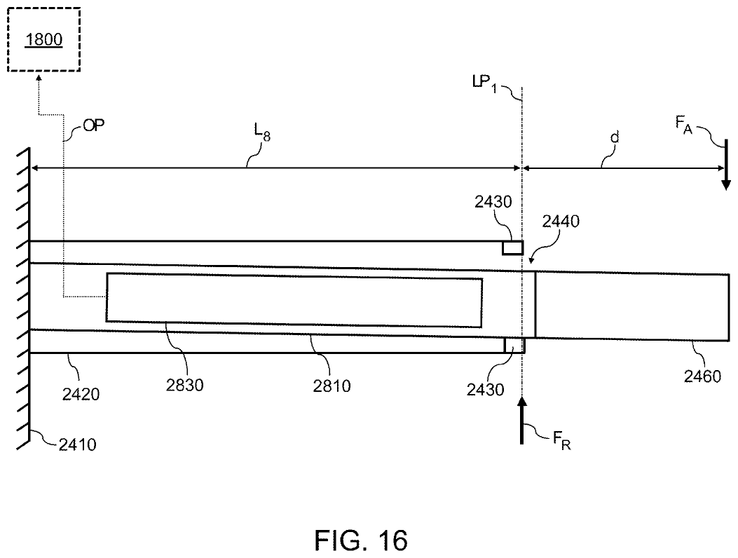

As described above, in embodiments in which the displacement of beam 2810 is limited by the hard stop 2430 and/or the cannula structure 2600, the strain distribution over the length of the beam 2810 may deviate relative to displacements of the beam 2810 that are not limited. Similarly stated, when the beam 2810 contacts the hard stop 2430, the beam 2810 no longer behaves as a cantilevered beam. In this condition, the deflection (δ) of the beam's distal end is larger than the maximum deflection permitted at the location of the hard stop 2430. As a result, the strain sensor(s) 2830 produce signals that do not accurately represent the applied force (F_{A}) affecting the end effector 2460, and so the force determined from the incorrect strain sensor signals may be significantly in error. For example, as shown in FIG. 15, when the applied force (F_{A}) increases to a magnitude that causes a deflection (δ) that results in the generation of the reactive force (F_{R}) (the initiation of which is depicted at point GPFR), the signals from the strain sensor(s) 2830 may result in a determined force (F) that is decreasing while the applied force (F_{A}) acting on the end effector 2460 is actually increasing (e.g., a force inversion condition may exist). Insofar as the controller 1800 may use the signals from the strain sensor(s) 2830 to generate the haptic feedback delivered to the surgeon (S), the inaccurate determination of the applied force (F_{A}) (i.e., an inaccurate determined force (F)) resulting in inaccurate haptic feedback is undesirable. It should therefore be appreciated that detecting such conditions of inaccurate determined force and mitigating the impact of inaccurate haptic feedback is beneficial to the operation of the surgical system 1000.

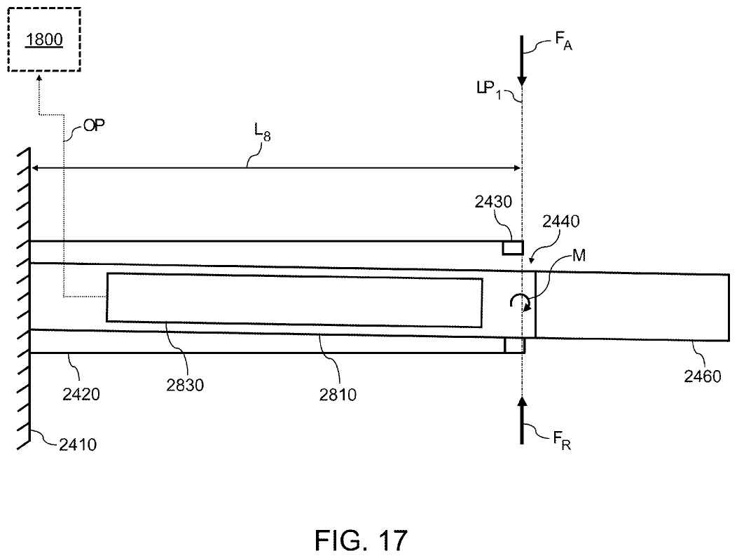

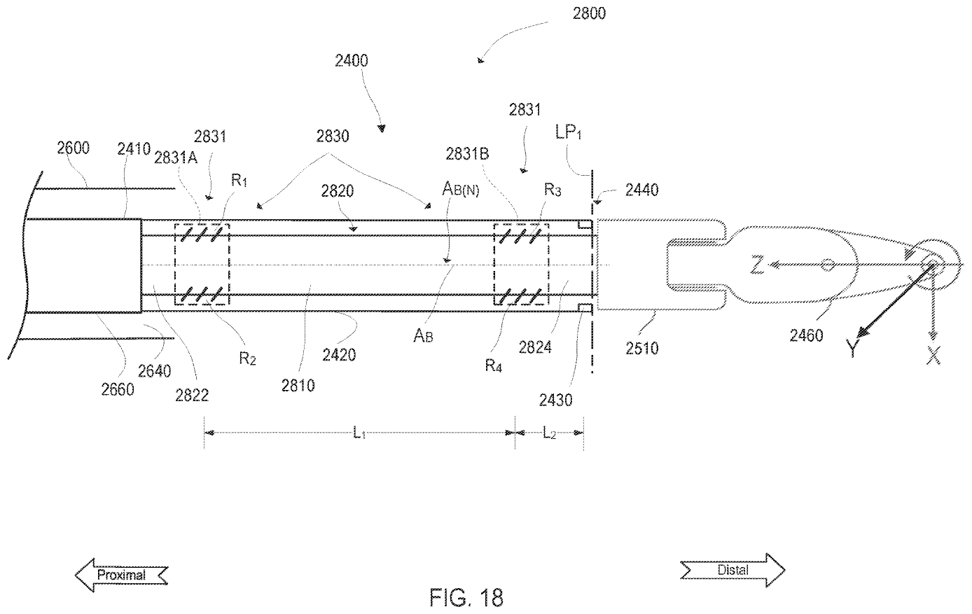

Accordingly, the controller 1800 (in connection with the arrangement of the strain gauge sensors R₁, R₂, R₃, and R₄) can be configured to (i) detect the occurrence of the condition in which the beam deflection (δ) is larger than a deflection threshold (Tδ) at which contact with the hard stop occurs, (ii) to provide an indication of this condition to the surgical system and/or the human clinical operator, and (iii) in some situations automatically take other actions in the surgical system. In accordance with an inventive aspect, this process is accomplished by determining a moment (M) at a reference location of the distal end portion of the instrument 2400 (see e.g., the reference location 2440 as indicated in FIG. 12). The moment (M) is a couple (i.e., a force couple, a pure moment, or a moment-of-couple) of the instrument 2400 according to rigid body mechanics (e.g., expressed in a free body diagram). Based at least on this moment (M), the deflection (δ) of the reference location can be estimated or otherwise determined. The following description, with reference to the free body diagrams of the instrument 2400 shown in FIGS. 16 and 17, provides details of the structure and methods for identifying the condition in which the deflection (δ) of the beam 2810 is larger than the deflection threshold (Tδ).

Specifically, in some embodiments the controller of the surgical system 1000, such as controller 1800, is configured to detect when the displacement of the beam 2810 is limited by the hard stop 2430 and thus mitigate any inaccurate determination of the applied force (F_{A}) and resulting haptic feedback to the human operator. Accordingly, the controller determines a moment (M) at a reference location 2440 of a distal end portion 2824 of the instrument 2400. The moment is, for example, determined based on the output of the strain sensor(s) 2830.

As depicted in FIGS. 11-13, in some embodiments the reference location 2440 is, in some embodiments, located at a longitudinal position (LP₁) along the medical instrument 2400. The longitudinal position (LP₁) is coplanar with the hard stop 2430. The reference location 2440 can be a portion of an outer surface of the beam 2810 that is opposite and facing the hard stop 2430. The reference location 2440 may, for example, be radially inward of the hard stop 2430 relative to the center axis (A_{B}).

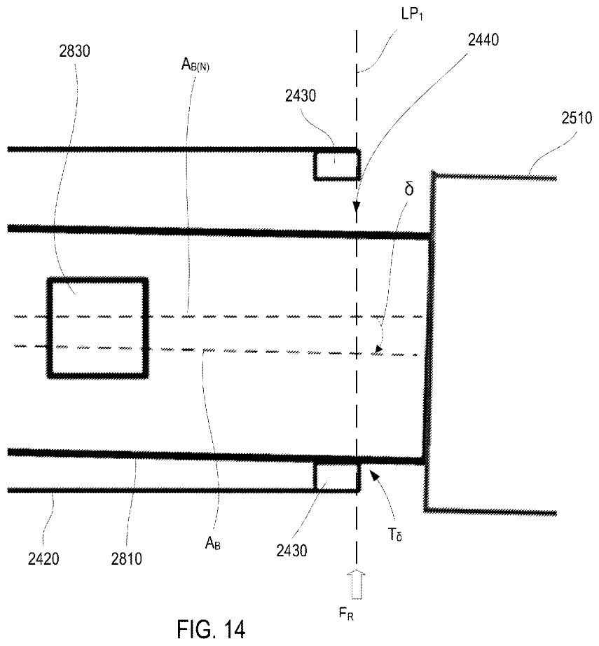

The positioning of the hard stop 2430 limits deflection (δ) of the reference location 2440. In other words, the distal end portion 2824 of the beam 2810 can bend relative to a proximal end portion 2822 of the beam 2810 such that the distal end portion 2824 of the beam 2810 is displaced a deflection distance relative to the designed position (A_{B}(N)) of the center axis (A_{B}) in response to an applied force (F_{A}). However, as depicted in FIGS. 13 and 14, this bending is limited when the beam 2810 encounters the hard stop 2430 due to the hard stop 2430 and/or the supporting structure being more rigid than the beam 2810.

FIG. 16 is a free-body diagram of the medical instrument shown in FIG. 11B, showing the applied force (F_{A}) developed at the end effector 2460. The applied force (F_{A}) can be developed by an interaction between the end effector 2460 and an object. For example, the applied force (F_{A}) can be developed by an interaction (e.g., pressing, pushing, pulling, and/or lifting) between the end effector 2460 and a portion of the body of the patient (P) or an object therein. As depicted, the applied force (F_{A}) is developed distally at a distance (d) from the reference location 2440. The reference location 2440 is cantilevered from a mechanical ground (e.g., the shaft 2410) by a beam length (L_{B}). FIG. 16 shows the beam 2810 in a deflected orientation in which the beam 2810 is in substantial contact with the hard stop 2430 of the shroud 2420. As a result of the contact between the beam 2810 in the hard stop 2430, a reactive force (F_{R}) in opposition to the applied force (F_{A}) is developed at the reference location 2440.

In order to detect a condition in which the displacement of the beam 2810 is limited by the hard stop 2430, it is desirable to resolve the applied force (F_{A}) at the reference location 2440 rather than at the end effector 2460. Accordingly, FIG. 17 is a free-body diagram of the medical instrument shown in FIG. 11B, showing the applied force (F_{A}) from FIG. 16 being resolved into an applied force (F_{A}) at the reference location 2440. Specifically, the force applied at the tip of the instrument (as shown in FIG. 16) can be resolved into the applied force (F_{A}) at the reference location 2440 and a moment (M), which is provided by Eq. (3):

$$M = {F}_{A} * d \qquad Eq . ( 3 )$$

Wherein the distance (d) is unknown, and the magnitude of the applied force (F_{A}) differs from the determined force (F) (as indicated by the strain sensor(s) 2830) in the presence of the reactive force (F_{R}) as illustrated in FIG. 17.

As depicted in FIGS. 16 and 17, the output (OP) of the strain sensor(s) 2830 correlates, at least in part, to the determined force (F). When the deflection (δ) of the beam 2810 in response to the applied force (F_{A}) does not result in the development of the reactive force (F_{R}) (e.g., when the beam 2810 does not substantially contact the hard stop 2430), the determined force (F) is effectively correlated to the applied force (F_{A}). But when the deflection (δ) of the beam 2810 in response to the applied force (F_{A}) places the beam 2810 in substantial contact with the hard stop 2430 (as shown in FIGS. 16 and 17), the determined force (F) is the result of a combination of the applied force (F_{A}) and the reactive force (F_{R}), which is provided by Eq. (4):

$$F = {F}_{A} - {F}_{R} \qquad Eq . ( 4 )$$

In accordance with Eq. (4), the magnitude of the determined force (F), as indicated by the strain sensor(s) 2830, is less than the magnitude of the actual applied force (F_{A}), which is unknown, due to the reactive force (F_{R}), which is also unknown, acting on the beam 2810 in the opposite direction when the beam 2810 is in substantial contact with the hard stop 2430.

As contact between the beam 2810 and the hard stop 2430 can affect force indications from the strain sensor(s) 2830, a deflection threshold (Tδ) is established in some embodiments. As depicted in FIG. 12, the deflection threshold (Tδ) can be established at a magnitude of deflection (δ) that is greater than zero but that precludes contact between an outer surface 2812 of the beam 2810 and a face 2432 (e.g., a radially inner face) of the hard stop 2430. For example, the deflection threshold (Tδ) can correspond to a first radial distance (RD₁) from the designed position (A_{B(N)}) of the center axis (A_{B}) that is less than a second radial distance (RD₂) (e.g., minimal radial distance) of the face 2432 of the hard stop 2430 from the designed position (A_{B(N)}) of the center axis (A_{B}). However, in some embodiments, the first radial distance (RD₁) can equal the second radial distance (RD₂) so that the deflection threshold (Tδ) corresponds to a deflection (δ) that places the outer surface 2812 in contact with the face 2432 but precludes the hard stop 2430 from exerting a reactive force on the beam 2810.

In some embodiments, the controller determines the deflection (δ) (e.g., the magnitude of the deflection) based on the moment (M). On a first condition in which the deflection (δ) is greater than a deflection threshold (Tδ), the controller provides an indication to the operator (S) of the input device, such as via the input device 1116, that a restriction of the haptic feedback is provided to, or is available to, be provided to the input device. The indication may be a visual indication, a haptic indication, and/or an audible indication. For example, the controller 1800 may, in an embodiment, be configured to generate a graphical indication of a deviation of the restricted haptic feedback from a designed haptic feedback (e.g., via the indicator module 1812). The controller 1800 may maintain (e.g., via the display system 1110) the graphical indication within a field-of-view 1142 (see FIG. 8) of the operator/surgeon (S) so long as the restriction of the haptic feedback is provided to, or is available to, be provided to the input device.

The moment (M), and ultimately the magnitude of the deflection (δ), is determined by the controller based on the output of the strain sensor(s) 2830. Specifically, by resolving the applied force (F_{A}) at the reference location and by configuring the strain sensors 2830 into multiple half-bridges (e.g., half-bridges 2831A and 2831B as depicted in FIG. 19), the resulting moment (M) can be determined as described below. The deflection (δ) of the beam 2810 can be determined, based on the moment (M) and the determined force (F) derived from the output of the strain sensors 2830. The relationship of the deflection (δ) to the moment (M) and the determined force (F) is provided by Eq. (5), where “E” is the modulus of elasticity of the beam 2810, “I” is the moment of inertia of the XY cross-section of the beam 2810, and “L_{B}” is the distance between the mechanical ground and the reference location 2440.

$$δ = \frac{F {L}_{B}^{3}}{3 E I} - \frac{M {L}_{B}^{2}}{2 E I} \qquad Eq . ( 5 )$$

In accordance with Eq. (5), when the beam 2810 is subjected to the applied force (F_{A}), the deflection of the beam 2810 can be determined based primarily on the physical characteristics of the beam 2810, the magnitude of the determined force (F) and measured moment (M). As such, an increase in the applied force (F_{A}) results in an increase in the deflection (δ). However, when the deflection (δ) is limited (e.g., stopped or resisted) by the hard stop 2430, a further increase in the applied force (F_{A}) is countered by a corresponding increase in the reactive force (F_{R}) (which results in the change in determined force (F) is less than the change in applied force (F_{A}), and in some cases even in the opposite). At the same time, the magnitude of the moment (M) increases based on the greater applied force (F_{A}) in accordance with Eq. (3). In such an instance, the deflection (δ) determined utilizing Eq. 5 can have a calculated magnitude that appears to be greater than the clearance between the beam 2810 and the hard stop 2430, and may, thus, indicate that the determined force (F) does not accurately represent the applied force (F_{A}).

To determine moment (M) and the determined force (F) at the reference location 2440, the controller is configured to receive indications of strain from the strain sensors 2830. As depicted in FIGS. 18 and 19, in some embodiments, the strain sensors 2830 include strain gauge resistors R₁, R₂, R₃, and R₄, which function as strain sensors and which can be arranged into one or more bridge circuits 2831 (e.g., a Wheatstone bridge). In such embodiments, the first strain gauge resistor (R₁) is configured to output a first strain indication (81). The relationship of the first strain indication (ε₁) to the determined force (F) and the moment (M) is described by Eq. (6):

$${ℰ}_{1} = \frac{F ( {L}_{1} + {L}_{2} ) r}{EI} + \frac{M r}{EI} - \frac{{F}_{Z}}{EA} \qquad Eq . ( 6 )$$

$$Where :$$

$$\begin{matrix}F = the determined force described by Eq . ( 4 ) \\ {L}_{1} = the distance between the first strain gauge resistor ( {R}_{1} ) and the third strain gauge resistor ( {R}_{3} ) \\ {L}_{2} = the distance between the third strain gauge resistor ( {R}_{3} ) and the reference location 2440 \\ r = one half the thickness of the beam 2810 \\ E = the modulus of elasticity of the beam 2810 \\ I = the moment of inertia of the XY cross - section of the beam 2810 \\ M = the moment described by Eq . ( 3 ) \\ {F}_{z} = a longitudinal force along the z - axis \\ A = area of a cross section of the beam 2810 in an X - Y plane\end{matrix}$$

The second strain gauge resistor (R₂) is configured to output a second strain indication (ε₂). The relationship of the second strain indication (ε₂) to the determined force (F) and the moment (M) is described by Eq. (7):

$${ℰ}_{2} = - \frac{F ( {L}_{1} + {L}_{2} ) r}{EI} - \frac{Mr}{EI} + \frac{{F}_{Z}}{EA} \qquad Eq . ( 7 )$$

The third strain gauge resistor (R₃) is configured to output a third strain indication (ε₃). The relationship of the third strain indication (ε₃) to the determined force (F) and the moment (M) is described by Eq. (8):

$${ℰ}_{3} = \frac{F {L}_{2} r}{EI} + \frac{M r}{EI} + \frac{{F}_{Z}}{EA} \qquad Eq . ( 8 )$$

The fourth strain gauge resistor (R₄) is configured to output a fourth strain indication (ε₄). The relationship of the fourth strain indication (ε₄) to the determined force (F) and the moment (M) is described by Eq. (9):

$${ℰ}_{4} = - \frac{F {L}_{2} r}{EI} - \frac{M r}{EI} + \frac{{F}_{Z}}{EA} \qquad Eq . ( 9 )$$

As depicted in FIGS. 18 and 19, the strain sensors 2830 can be arranged into at least two half-bridge circuits 2831. Each half-bridge circuit 2831A, 2831B can include a portion of the strain gauge resistors R₁, R₂, R₃, and R₄. In some embodiments, the half-bridge circuits 2831 can be arranged along a single face of the beam 2810. However, in additional embodiments, the half-bridge circuits 2831 can be arranged along adjacent, separated, or opposing faces of the beam 2810. Additionally, as depicted in FIG. 19, in some embodiments, a first half-bridge circuit 2831A includes proximally positioned strain gauge resistors (e.g., strain gauge resistors R₁ and R₂), while the second half-bridge circuit 2831B includes distally positioned strain gauge resistors (e.g., strain gauge resistors R₃ and R₄). However, in additional embodiments, each half-bridge circuit 2831 can include at least one proximally positioned strain gauge resistor and at least one distally positioned strain gauge resistor.

FIG. 19 is a diagrammatic illustration of one configuration of the strain sensors 2830, showing the first half-bridge circuit 2831A and the second half-bridge circuit 2831B. The first half-bridge circuit 2831A can include the first strain gauge resistor (R₁) and the second strain gauge resistor (R₂) and can be electrically coupled to at least one precision resistor (RP). The precision resistor(s) (RP) is configured as a reference resistor and may have a fixed resistive value or an adjustable resistive value (e.g., a potentiometer). The second half-bridge circuit 2831B can include the third strain gauge resistor (R₃) and the fourth strain gauge resistor (R₄) and can be electrically coupled to the precision resistor(s) (RP). In order to detect strain, an input voltage (VIN) is provided to the first half-bridge circuit 2831A and/or the second half-bridge circuit 2831B. A first output voltage (V_{A}) can then be measured for the first half-bridge circuit 2831A. The first output voltage (V_{A}) corresponds to a first half-bridge strain indication (ε₁-ε₂) as described by Eq. (10). A second output voltage (V_{B}) can then be measured for the second half-bridge circuit 2831B. The second output voltage (V_{B}) corresponds to a second half-bridge strain indication (ε₃-ε₄) as described by the Eq. (11).

$$( {ℰ}_{1} - {ℰ}_{2} ) ∝ {V}_{A} \qquad Eq . ( 10 )$$

$$( {ℰ}_{3} - {ℰ}_{4} ) ∝ {V}_{B} \qquad Eq . ( 11 )$$

As the first half-bridge strain indication (ε₁-ε₂) is proportional to the measured first output voltage (V_{A}), the relationship of the first half-bridge strain indication (ε₁-ε₂) to the determined force (F) and the moment (M) can be determined by combining the strain equations for the corresponding strain gauge resistors, specifically, Eq. (6) and Eq. (7). The strain equation for the first half-bridge strain indication (ε₁-ε₂) resolves to Eq. (12):

$$( {ℰ}_{1} - {ℰ}_{2} ) = 2 ( \frac{F ( {L}_{1} + {L}_{2} ) r}{EI} + \frac{Mr}{EI} ) \qquad Eq . ( 12 )$$

The resolving of Eq. (6) and Eq. (7) eliminates the longitudinal force (Fz) along the z-axis. However, magnitudes of the determined force (F) and moment (M) remain unknown.

Similarly, as the second half-bridge strain indication (ε₃-ε₄) is proportional to the measured second output voltage (V_{B}), the relationship of the second half-bridge strain indication (ε₃-ε₄) to the determined force (F) and the moment (M) can be determined by combining the strain equations for the corresponding strain gauge resistors, specifically, Eq. (8) and Eq. (9). The strain equation for the second half-bridge strain indication (ε₃-ε₄) resolves to Eq. (13):

$$( {ℰ}_{3} - {ℰ}_{4} ) = 2 ( \frac{F {L}_{2} r}{EI} + \frac{M r}{EI} ) \qquad Eq . ( 13 )$$

The resolving of Eq. (8) and Eq. (9) eliminates the longitudinal force (Fz) along the z-axis, with the magnitudes of the determined force (F) and moment (M) remain unknown.

With the strain sensor 2830 arranged as at least two half-bridge circuits 2831, and the applied force (F_{A}) resolved at the reference location 2440, the controller is configured to employ the measured voltage outputs (e.g., the first output voltage (V_{A}) and the second output voltage (V_{B})) of the half-bridge circuits 2831 to determine the magnitudes of the determined force (F) and moment (M). Specifically, the controller is configured to determine the magnitudes of the determined force (F) and moment (M) by solving the two strain equations (e.g., Eq. (12) and Eq. (13)) for the two unknown variables based on the measured first output voltage (V_{A}) and the measured second output voltage (V_{B}).

Following the determination of the magnitudes of the determined force (F) and moment (M) by solving the strain equations for the half-bridge circuits 2831 (e.g., Eq. (12) and Eq. (13)) based on the measured output voltages of the half-bridge circuits 2831, the controller is configured to determine the deflection (δ) of the beam 2810. Specifically, the controller is configured to utilize the determined magnitudes of the determined force (F) and moment (M) to determine the deflection (δ) of the beam 2810 based on the relationship of the deflection (δ) to the moment (M) and the determined force (F) is described by Eq. (5).

On a first condition in which the deflection (δ) is greater than the deflection threshold (Tδ), the controller provides the indication to the operator (S) of the surgical system (e.g., the surgical system 1000) that the restriction of the haptic feedback is provided to, or is available to, be provided to the input device. In some embodiments, the restriction of the haptic feedback corresponds to a halting of an operation of the surgical system. For example, when the deflection (δ) is greater than the deflection threshold (Tδ), the controller may halt a movement of the medical instrument 2400 such that the end effector 2460 is maintained in a fixed position until the restriction of the haptic feedback is acknowledged by the operator (S).

After the first condition, and on a second condition in which the deflection (δ) is less than the deflection threshold (Tδ), the controller can remove the restriction of the haptic feedback. In other words, when the magnitude of the applied force (F_{A}) is reduced to a point at which the deflection (δ) of the beam 2810 returns to a magnitude that is less than the deflection threshold (Ta), the determined force (F) equals the applied force (F_{A}) and unrestricted haptic feedback can be provided to the operator (S).

During the first condition, the controller is, in some embodiments, configured to determine an estimated applied force (F_{A}). The estimated applied force (F_{A}) can be based on the moment (M). Specifically, the estimated applied force (F_{A}) is presumed to be developed distally at a distance (d) from the reference location 2440. In order to generate the estimated applied force (F_{A}), the distance (d) is presumed to correspond to the distance between the reference location 2440 and a designated position along the end effector 2460, such as a contact surface of a tool member. The estimated applied force (F_{A}) can then be determined by dividing the moment (M) (determined based on the output of the strain sensors 2830) by the presumed distance (d), as described by Eq. (3). The presumed distance (d) can be calculated when the deflection is less than the deflection threshold (Tδ), by simply dividing the moment (M) by the determined force (F), which should be equal (F_{A}). In other words when the beam 2410 is not in hard stop contact the loading location (e.g., the presumed distance (d)) can be estimated. When the beam 2410 is in hard stop contact the last estimated loading distance can be used to estimate the applied force (F_{A}).

In some embodiments, the estimated applied force (F_{A}) can be determined based on an estimated reactive force (F_{R}) developed by the interaction between the beam 2810 and the hard stop 2430. To determine the estimated reactive force (F_{R}), the controller determines an indicated deflection (δ) based on the moment (M) and the determined force (F) in accordance with Eq. (5). However, the clearance between the between the hard stop 2430 and the beam 2810 (e.g., the second radial distance (RD₂) as depicted in FIG. 12) establishes a maximal deflection for the beam 2810. As such, the portion of the indicated deflection (δ) that would otherwise exceed the maximal deflection corresponds to a resisted deflection portion. The controller is then configured to determine the estimated reactive force (F_{R}) by multiplying the magnitude of the resisted deflection portion by a stiffness factor (e.g., a spring constant) for the hard stop 2430. Finally, the estimated reactive force (F_{R}) can be subtracted from the determined force (F) to determine the applied force (F_{A}) in accordance with Eq. (4).

In some embodiments, the controller is configured to execute an operation of the surgical system based on the estimated applied force (F_{A}). For example, the controller can deliver a haptic feedback magnitude to the operator (S) of the input device (e.g., the input device 1116) of the surgical system that corresponds to the estimated applied force (F_{A}). In additional embodiments, the execution of the operation of the surgical system based on the estimated applied force (F_{A}) can, for example, include limiting movement of the medical instrument 2400, executing a predefined movement of the medical instrument 2400, and/or executing a load-mitigation operation. By way of illustration, in some embodiments, the controller can halt and operation surgical system when the estimated applied force (F_{A}) exceeds a threshold value.

In some embodiments, the restricted haptic feedback corresponds to a full restriction of a designed haptic feedback. In such embodiments, the magnitudes of the restricted haptic feedback along each axis are each less than corresponding designed haptic feedback magnitudes. However, in some embodiments, the restricted haptic feedback corresponds to a partial restriction of the designed haptic feedback. For example, in such embodiments, the magnitudes of the restricted haptic feedback HF_{R} along one axis may be less than the corresponding designed haptic feedback magnitude while magnitudes along the other axes are unaffected.

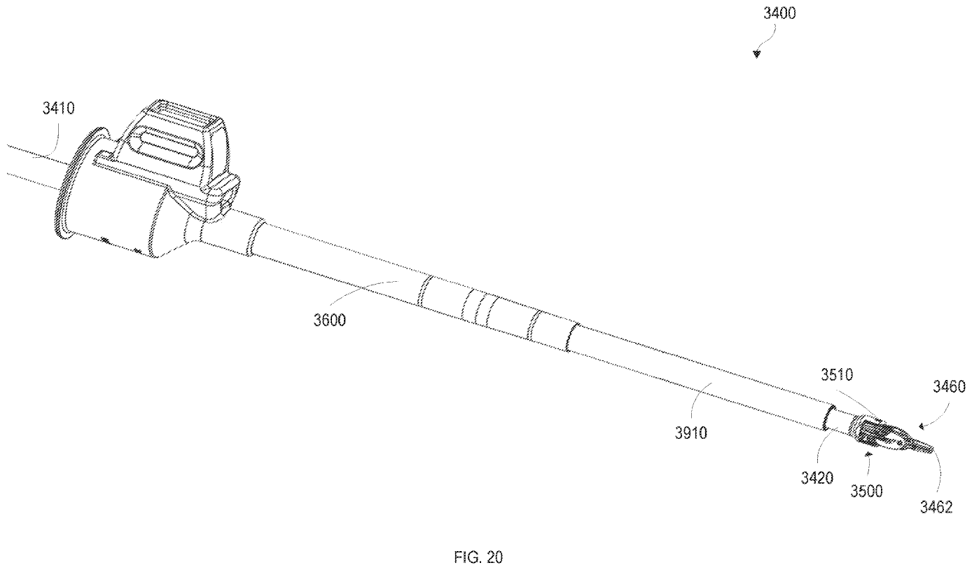

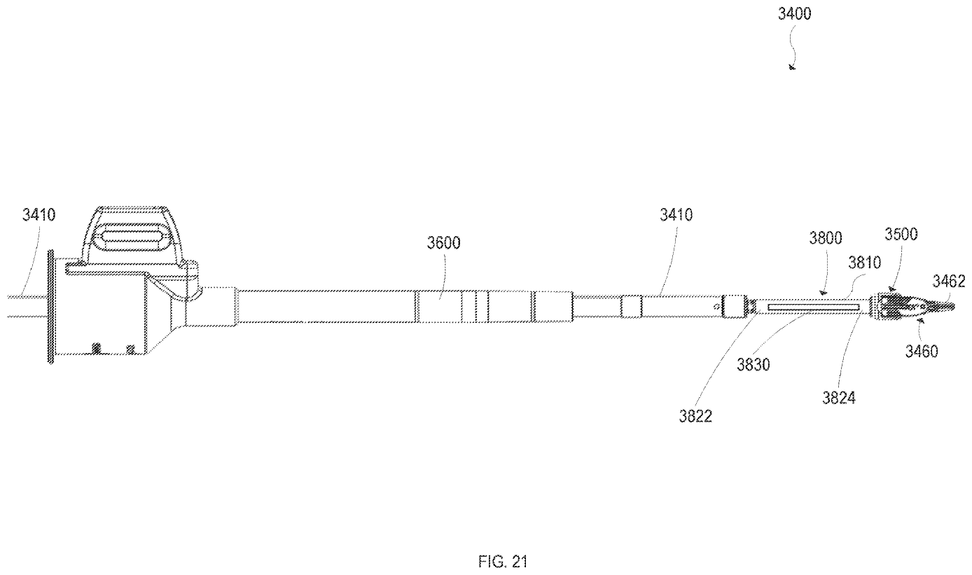

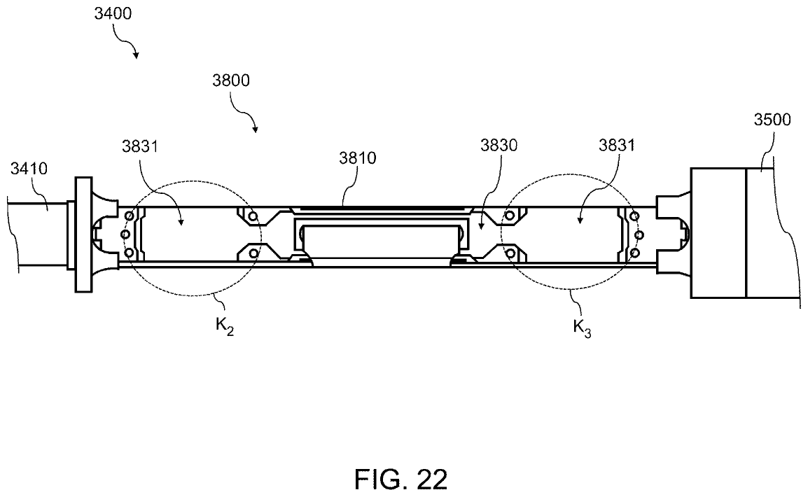

FIGS. 20 and 21 depict a perspective view and a side view (with the outer shaft and shroud removed for clarity) of a medical instrument 3400 and a cannula 3600, while FIGS. 22-26 depict additional views and enlargements of portions thereof of a force sensor unit 3800 of the instrument 3400. In some embodiments, the instrument 3400 or any of the components therein are optionally parts of a surgical system that performs surgical procedures. The surgical system may include a manipulator unit, a series of kinematic linkages, a series of cannulas, or the like. The instrument 3400 (and any of the instruments described herein) can be used in any suitable surgical system, such as the MIRS system 1000 shown and described above.

The instrument 3400 includes a proximal mechanical structure (not shown), an outer shaft 3910, a shaft 3410, a force sensor unit 3800 that includes a beam 3810, a wrist assembly 3500, and an end effector 3460. As depicted, in an embodiment, a shroud 3420 may circumscribe at least a portion of the beam 3810. Although not shown, the instrument 3400 can also include a number of cables that couple the mechanical structure to the wrist assembly 3500 and end effector 3460. The instrument 3400 is configured such that select movements of the cables produces rotation of the wrist assembly 3500 (i.e., pitch rotation) about an axis of rotation (which functions as a pitch axis, the term pitch is arbitrary), yaw rotation of the end effector 3460 about an additional axis of rotation (which functions as the yaw axis, the term yaw is arbitrary), a cutting rotation of the tool members of the end effector 3460, or any combination of these movements. Changing the pitch or yaw of the instrument 3400 can be performed by manipulating the cables in a similar manner as described, for example, in U.S. Pat. No. 8,821,480 B2 (filed Jul. 16, 2008), entitled “Four-Cable Wrist with Solid Surface Cable Channels,” which is incorporated herein by reference in its entirety.

In some embodiments, the end effector 3460 can include at least one tool member 3462 having a contact portion configured to engage or manipulate a target tissue during a surgical procedure. For example, in some embodiments, the contact portion can include an engagement surface that functions as a gripper, cutter, tissue manipulator, or the like. In other embodiments, the contact portion can be an energized tool member that is used for cauterization or electrosurgical procedures. The end effector 3460 may be operatively coupled to the proximal mechanical structure such that the tool member 3462 rotates relative to shaft 3410. In this manner, the contact portion of the tool member 3462 can be actuated to engage or manipulate a target tissue during a surgical procedure. The tool member 3462 (or any of the tool members described herein) can be any suitable medical tool member. Moreover, although only one tool member 3462 is identified, as shown, the instrument 3400 can include two tool members that cooperatively perform gripping or shearing functions. In other embodiments, an end effector can include more than two tool members.

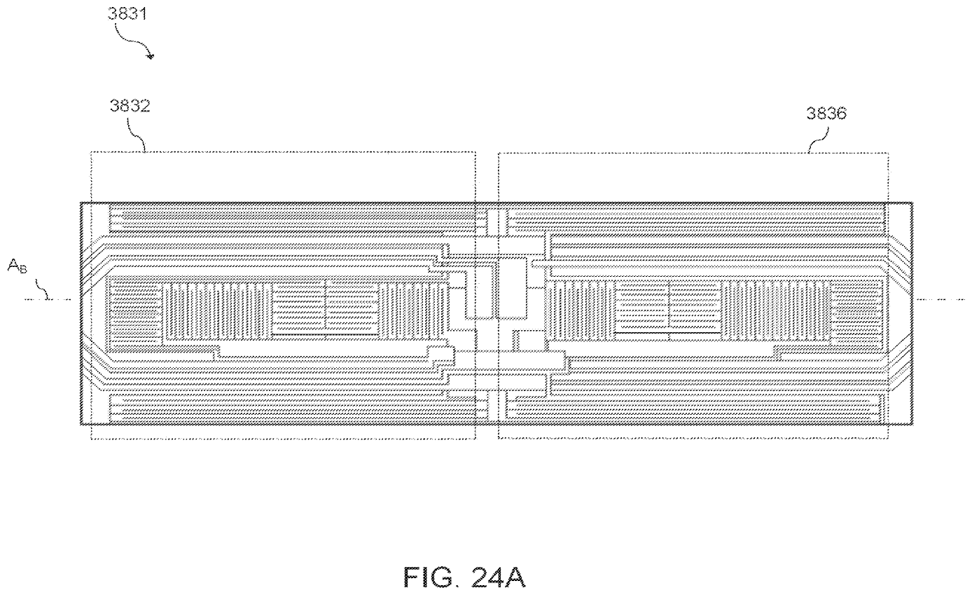

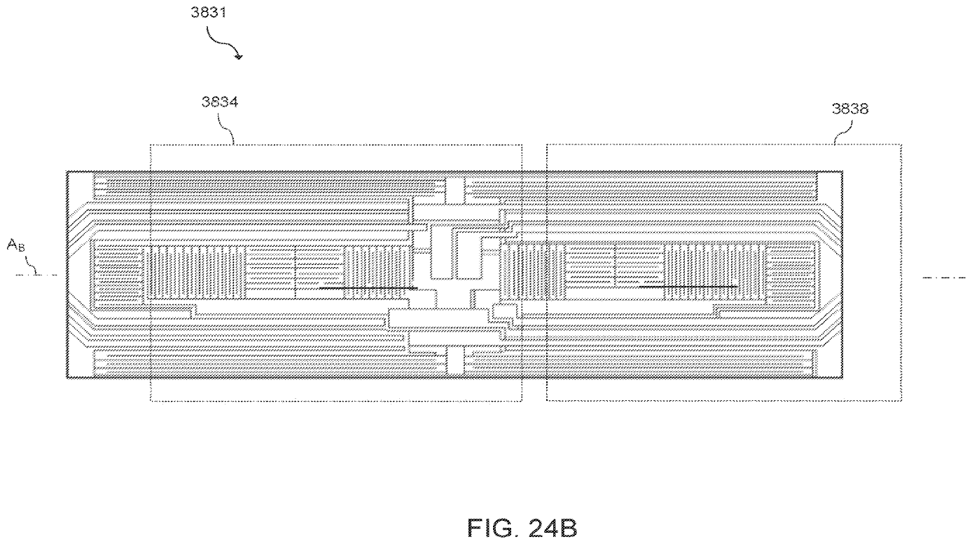

In some embodiments, the force sensor unit 3800 includes one or more strain sensors 3830 mounted on the beam 3810. The strain sensors 3830 can be, for example, strain gauges, and may be used to measure forces imparted on the surgical instrument during a surgical procedure as described in more detail herein. In some embodiments, the beam 3810 may define at least three side surfaces disposed acutely to each other. In additional embodiments, the beam 3810 may define at least four side surfaces disposed perpendicular to one another. The strain sensor(s) 3830 may be mounted to the side surfaces in appropriate locations. The beam 3810 defines a beam center axis (A_{B}) (see FIGS. 24A-26) which can be aligned within a center axis (not shown) of the instrument shaft 3410. The beam center axis (A_{B}) is a neutral axis that is equidistant from the sides (e.g., faces) of the beam 3810.

In use, the end effector 3460 may contact anatomical tissue, which may result in X, Y, or Z direction forces (similar to the forces exerted on the end effector 2460 shown in FIG. 13) being imparted on the end effector 3460. This contact may also result in forces about the various axes. The strain sensors 3830 may be used to measure strain in the beam 3810 as a result of such forces imparted on the end effector 3460. More specifically, the strain sensors 3830 can measure forces imparted on the end effector 3460 that are transverse (e.g., perpendicular) to a center axis of the beam 3810 as such forces are transferred to the beam 3810 in the X and Y directions (see FIG. 11B). Specifically, the transverse forces acting upon the end effector 3460 can cause a slight bending of the beam 3810, which can result in a tensile strain imparted to one side of the beam 3810 and a compression strain imparted to an opposing side of the beam 3810. The strain sensors 3830 may be coupled to the beam 3810 to measure such tensile and compression forces, with the resultant measurements being communicated to the controller via a communication coupling therebetween.

More specifically, when a force is imparted on a distal portion of the medical instrument 3400 (e.g., at end effector 3460) in the X or Y directions (see FIG. 11B for reference to X, Y and Z directions), such transverse force can cause the beam 3810 to bend (about either or some combination of the X axis or the Y axis), which can result in a tensile strain imparted to one side of the beam 3810 and a compression strain imparted to the opposite side of the beam 3810. The strain sensors 3830 on the beam 3810 can measure such tensile and compression strains.

In some embodiments, the force sensor unit 3800 includes the beam 3810, with one or more bridge circuits 3831 (see, e.g., FIGS. 24-26), which can form one or more strain sensors 3830 (which can be Wheatstone bridges) mounted on a surface along the beam 3810. As described herein, each bridge circuit 3831 (and also each strain sensor) can include one or more strain gauges (e.g., tension strain gauge resistor(s) or compression strain gauge resistor(s). In some embodiments, a shroud 3420 may circumscribe at least a portion of the beam 3810, and an end effector 3460 may by coupled at a distal end portion 3824 of the beam 3810 (e.g. at a distal end portion of the surgical instrument 3400). The end effector 3460 can include, for example, articulatable jaws, a cautery instrument, and/or any other suitable surgical tool 3462 that is coupled to a link 3510 (e.g., a proximal clevis pin). In some embodiments, the link 3510 can be included within a wrist assembly having multiple articulating links. In some embodiments the link 3510 is included as part of the end effector 3460. The shaft 3410 includes a distal end portion that is coupled to a proximal end portion 3822 of the beam 3810. In some embodiments, the distal end portion of the shaft 3410 is coupled to the proximal end portion 3822 of the beam via another coupling component (such as an anchor or coupler, not shown). The shaft 3410 can also be coupled at a proximal end portion to a mechanical structure (not shown) configured to move one or more components of the surgical instrument, such as, for example, the end effector 3460.

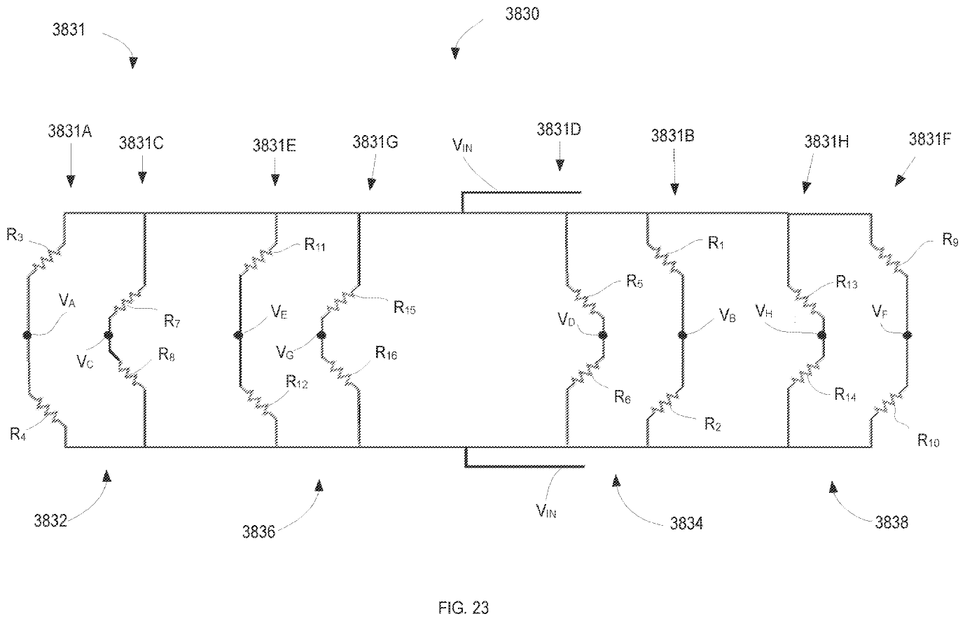

Referring to FIGS. 23-26, the strain gauge resistors (such as strain gauge resistors R₁, R₂, R₃, R₄, R₅, R₆, R₇, R₈, R₉, R₁₀, R₁₁, R₁₂, R₁₃, R₁₄, R₁₅, and R₁₆ (R₁₋₁₆) which form portions of the strain sensors and which can be arranged into one or more bridge circuits 3831 (e.g., one or more Wheatstone bridges), can measure strain in the beam 3810 that can be used to determine the forces imparted on the end effector 3460 in the X and Y axes directions (See FIG. 18) according to any of the methods described herein (including the methods described above with reference to the instrument 2400). These X and Y axes forces are transverse (e.g., perpendicular) to the Z axis (which is parallel or collinear with the longitudinal center axis (A_{B}) of the beam 3810). Such transverse forces acting upon the end effector 3460 can cause a deflection (e.g. bending) of the beam 3810 (about either or both of the X axis or the Y axis), which can result in a tensile strain imparted to one side of the beam 3810 and a compression strain imparted to the opposite side of the beam 3810. The strain gauge resistors R₁₋₁₆ on the beam 3810 may measure such tensile and compression strains. The output of the strain gauge resistors R₁₋₁₆ on the beam 3810 may be corelated to a determined force (see, e.g., FIG. 17). Moreover, as described herein, depending on the arrangement of the strain gauge resistors R₁₋₁₆ (i.e., in at least two bridge circuits 3831 (e.g., eight half-bridges 3831), the strain gauge resistors R₁₋₁₆ can measure the moment. It should be appreciated that the output of the force sensor unit 3800 may be utilized by a controller, such as the controller 1800 of system 1000 described above, to determine the haptic feedback to deliver to the surgeon (S) via the input control device(s) 1116.

Although shown as including only the force sensor unit 3800, in some embodiments, the instrument 3400 (or any of the instruments described herein) can include additional force sensor units to measure the axial force(s) (i.e., in the direction of the Z-axis parallel to the beam center axis (A_{B})) imparted on the end effector 3460. An axial force sensor unit in an example surgical instrument can comprise a deflectable planar diaphragm sensor that deflects in response to a force. Alternatively, a deflectable ferrite core can be used within an inductive coil may be used or a or a fiber Bragg grating formed within an optical fiber can be used, for example. Other axial force sensor units may be used to sense a resilient axial displacement of the shaft 3410 (e.g., relative to the proximally mounted mechanical structure, not shown). An axial force Fz imparted to the end effector 3460 can cause axial displacement of the shaft 3410 in a direction along a center axis of the shaft (substantially parallel to the beam center axis (A_{B})). The axial force Fz may be in the proximal direction (e.g., a reactive force resulting from pushing against tissue with the end effector) or it may be in the distal direction (e.g., a reactive force resulting from pulling tissue grasped with the end effector).

In some embodiments, X and/or Y forces imparted on the end effector 3460 may result in strain in the beam 3810 when the beam 3810 is deflected (e.g., displaced or bent). In other words, the X and/or Y forces deflect the center axis (A_{B}) of the beam 3810 away from a designed position (A_{B(N)}) (similar to the deflection shown for the beam 2810 shown in FIG. 14) of the center axis (A_{B}), and, thus, relative to a center axis of the shaft 3410. Said another way, a distal end portion 3824 of the beam 3810 can bend relative to a proximal end portion 3822 of the beam 3810 such that the end portion 3824 of the beam 3810 is displaced a deflection distance relative to the designed position (A_{B(N)}) of the center axis (A_{B}).

In some embodiments, the shroud 3420 and/or the cannula structure 3600 can limit the displacement of the beam 3810 and produce a reactive force that is exerted on the beam 3810. For example, the shroud 3420 can include or function as a hard stop (e.g., similar to the hard stop 2430 as depicted in FIGS. 11-14). In an embodiment wherein the displacement of beam 3810 is limited by the hard stop and/or the cannula structure 3600, the strain distribution over the length of the beam 3810 may deviate relative to displacements of the beam 3810 that are not limited. Similarly stated, when the beam 3810 contacts the hard stop, the beam 3810 no longer behaves as a cantilevered beam. In this condition, the deflection is greater than the maximum deflection permitted at the location of the hard stop. As a result, the strain sensor(s) 3830 may produce signals that do not accurately represent the applied force affecting the end effector 3460. For example, when the applied force causes a deflection that results in the generation of the reactive force, the signals from the strain sensor(s) 3830 may indicate a force that is decreasing while the applied force acting on the end effector 3460 is actually increasing (e.g., a force inversion condition may exist). Insofar as the controller 1800 may utilize the signals from the strain sensor(s) 3830 to generate the haptic feedback delivered to the surgeon (S), the inaccurate representation of the applied force resulting in inaccurate haptic feedback may be undesirable. It should therefore be appreciated that detecting such conditions and mitigating the impact of inaccurate haptic feedback may be beneficial to the operation of the surgical system 1000.

Accordingly, the controller 1800 (in connection with the arrangement of the strain gauge sensors R₁₋₁₆ as more fully described below) can be configured to implement any of the methods and procedures described herein. Specifically, the controller can utilize the force sensor unit 3800 to detect the occurrence of the condition in which the deflection of the beam is greater than a deflection threshold and provide an indication of this condition (and, in some situations, take other actions). This is accomplished by determining a moment at a reference location of the distal end portion of the instrument 3400 (see e.g., the reference location 2440 as indicated in FIG. 12). Based on this moment, the deflection of the reference location can be estimated or otherwise determined.

As depicted in FIGS. 22-26, the strain sensors 3830 can be arranged into at least two half-bridge circuits 3831 (e.g., eight half-bridge circuits 3831(A-H) as depicted in FIG. 24). Each half-bridge circuit 3831 can include a portion of the strain gauge resistors R₁₋₁₆ and can be electrically coupled to at least one precision resistor (not shown). The precision resistor(s) is configured as a reference resistor and may have a fixed resistive value or an adjustable resistive value (e.g., a potentiometer). In some embodiments, the half-bridge circuits 3831 can be arranged along a single face of the beam 3810 as depicted in FIG. 22. However, in additional embodiments, the half-bridge circuits 3831 can be arranged along adjacent, separated, or opposing faces of the beam 3810. Additionally, as depicted in FIG. 24A, in some embodiments, a first half-bridge circuit 3831A includes proximally positioned strain gauge resistors (e.g., strain gauge resistors R₃ and R₄), while the second half-bridge circuit 3831B depicted in FIG. 24B includes distally positioned strain gauge resistors (e.g., strain gauge resistors R₁ and R₂). However, in additional embodiments, each half-bridge circuit 3831 can include at least one proximally positioned strain gauge resistor and at least one distally positioned strain gauge resistor.

FIG. 23 is a diagrammatic illustration of one configuration of the strain sensor 3830, showing eight half-bridge circuits 3831A-3831H. The eight half-bridge circuits 3831 include the first half-bridge circuit 3831A, the second half-bridge circuit 3831B, a third half-bridge circuit 3831C, a fourth half-bridge circuit 3831D, a fifth half-bridge circuit 3831E, a sixth half-bridge circuit 3831F, a seventh half-bridge circuit 3831G, and an eighth half-bridge circuit 3831H. In order to detect strain, an input voltage (VIN) is provided to the eight half-bridge circuits 3831(A-H), and an output voltage (e.g., V_{A}, V_{B}, V_{C}, V_{D}, V_{E}, V_{F}, V_{G}, and V_{H} (V_{A-H})) can then be measured for each of the eight half-bridge circuits 3831(A-H). Various combinations of the output voltages (V_{A-H}) may be employed by the controller as previously described to determine the measure-force and the moment.

In some embodiments, the first half-bridge circuit 3831A and the third half-bridge circuit 3831C are arranged as a primary proximal bridge-circuit combination 3832, while the second half-bridge circuit 3831B and the fourth half-bridge circuit 3831D are arranged as a primary distal bridge-circuit combination 3834. Additionally, in some embodiments, the fifth half-bridge circuit 3831E and the seventh half-bridge circuit 3831G are arranged as a secondary proximal bridge-circuit combination 3836, while the sixth half-bridge circuit 3831F and the eighth half-bridge circuit 3831H are arranged as the secondary distal bridge-circuit combination 3838. An output of the secondary proximal bridge-circuit combination 3836 is redundant to a corresponding output of the primary proximal bridge-circuit combination 3832. Similarly, an output of the secondary distal bridge-circuit combination 3838 is redundant to a corresponding output of the primary distal bridge-circuit combination 3834. In other words, absent a sensor malfunction, the outputs of the secondary proximal bridge-circuit combination 3836 and the secondary distal bridge-circuit combination 3838 equal the outputs of the primary proximal bridge-circuit combination 3832 and the primary distal bridge-circuit combination 3834.

As depicted in FIGS. 23 and 24A, the first half-bridge circuit 3831A can include the third strain gauge resistor (R₃) and the fourth strain gauge resistor (R₄). As depicted in FIG. 24A, the third and fourth strain gauge resistors (R₃, R₄) can be positioned on opposite sides of the beam center axis (A_{B}) and equidistant from the center axis (A_{B}). For example, the third and fourth strain gauge resistors (R₃, R₄) can be positioned equidistant between the beam center axis (A_{B}) and a side edge of the surface to which they are mounted. In some embodiments, the third and fourth strain gauge resistors (R₃, R₄) can be positioned at the same proximal position along the beam center axis (A_{B}). As depicted, in some embodiments, the third and fourth strain gauge resistors (R₃, R₄) are both the same type of strain gauge resistor (e.g., are both tension strain gauge resistors).

As further depicted in FIGS. 23 and 24A, the third half-bridge circuit 3831C can include the seventh strain gauge resistor (R₇) and the eighth strain gauge resistor (R₈). As depicted in FIG. 24A, the seventh and eighth strain gauge resistors (R₇, R₈) are positioned in axial alignment with the beam center axis (A_{B}). In some embodiments, a portion of the eighth strain gauge resistor (R₈) is positioned axially between the portions of the seventh strain gauge resistor (R₇), and a portion of the seventh strain gauge resistor (R₇) is positioned axially between the portions of the eighth strain gauge resistor (R₈). As depicted, one of the seventh and eighth strain gauge resistors (R₇, R₈) is a tension strain gauge resistor while the other is a compression strain gauge resistor.

As depicted in FIGS. 23 and 24B, the second half-bridge circuit 3831B can include the first strain gauge resistor (R₁) and the second strain gauge resistor (R₂). As depicted in FIG. 24B, the first and second strain gauge resistors (R₁, R₂) can be positioned on opposite sides of the beam center axis (A_{B}) and equidistant from the center axis (A_{B}). For example, the first and second strain gauge resistors (R₁, R₂) can be positioned equidistant between the beam center axis (A_{B}) and a side edge of the surface to which they are mounted. In some embodiments, the first and second strain gauge resistors (R₁, R₂) can be positioned at the same proximal position along the beam center axis (A_{B}). As depicted, in some embodiments, the first and second strain gauge resistors (R₁, R₂) are both the same type of strain gauge resistor (e.g., are both tension strain gauge resistors).

As further depicted in FIGS. 23 and 24B, the fourth half-bridge circuit 3831D can include the fifth strain gauge resistor (R₅) and the sixth strain gauge resistor (R₆). As depicted in FIG. 24B, the fifth and sixth strain gauge resistors (R₅, R₆) are positioned in axial alignment with the beam center axis (A_{B}). In some embodiments, a portion of the sixth strain gauge resistor (R₆) is positioned axially between the portions of the fifth strain gauge resistor (R₅), and a portion of the fifth strain gauge resistor (R₅) is positioned axially between the portions of the sixth strain gauge resistor (R₆). As depicted, one of the fifth and sixth strain gauge resistors (R₅, R₆) is a tension strain gauge resistor while the other is a compression strain gauge resistor.

Referring again to FIGS. 23 and 24A, as depicted, the fifth half-bridge circuit 3831E can include the eleventh strain gauge resistor (R₁) and the twelfth strain gauge resistor (R₁₂). As depicted in FIG. 24A, the eleventh and twelfth strain gauge resistors (R₁, R₁₂) can be positioned on opposite sides of the beam center axis (A_{B}) and equidistant from the center axis (A_{B}). For example, the eleventh and twelfth strain gauge resistors (Rn, R₁₂) can be positioned equidistant between the beam center axis (A_{B}) and a side edge of the surface to which they are mounted. In some embodiments, the eleventh and twelfth strain gauge resistors (R₁, R₁₂) can be positioned at the same proximal position along the beam center axis (A_{B}). As depicted, in some embodiments, the eleventh and twelfth strain gauge resistors (R₁, R₁₂) are both the same type of strain gauge resistor (e.g., are both tension strain gauge resistors). The fifth half-bridge circuit 3831E is positioned distally relative to the first half-bridge circuit 3831A.

As further depicted in FIGS. 23 and 24A, the seventh half-bridge circuit 3831G can include the fifteenth strain gauge resistor (R₁₅) and the sixteenth strain gauge resistor (R₁₆). As depicted in FIG. 24A, the fifteenth and sixteenth strain gauge resistors (R₁₅, R₁₆) are positioned in axial alignment with the beam center axis (A_{B}). In some embodiments, a portion of the fifteenth strain gauge resistor (R₁₅) is positioned axially between the portions of the sixteenth strain gauge resistor (R₁₆), and a portion of the sixteenth strain gauge resistor (R₁₆) is positioned axially between the portions of the fifteenth strain gauge resistor (R₁₅). As depicted, one of the fifteenth and sixteenth strain gauge resistors (R₁₅, R₁₆) is a tension strain gauge resistor while the other is a compression strain gauge resistor. The seventh half-bridge circuit 3831G is positioned distally relative to the third half-bridge circuit 3831C.

As depicted in FIGS. 23 and 24B, the sixth half-bridge circuit 3831F can include the 9^{th} strain gauge resistor (R₉) and the tenth strain gauge resistor (R₁₀). As depicted in FIG. 24B, the ninth and tenth strain gauge resistors (R₉, R₁₀) can be positioned on opposite sides of the beam center axis (A_{B}) and equidistant from the center axis (A_{B}). For example, the ninth and tenth strain gauge resistors (R₉, R₁₀) can be positioned equidistant between the beam center axis (A_{B}) and a side edge of the surface to which they are mounted. In some embodiments, the ninth and tenth strain gauge resistors (R₉, R₁₀) can be positioned at the same proximal position along the beam center axis (A_{B}). As depicted, in some embodiments, the ninth and tenth strain gauge resistors (R₉, R₁₀) are both the same type of strain gauge resistor (e.g., are both tension strain gauge resistors). The sixth half-bridge circuit 3831F is positioned distally relative to the second half-bridge circuit 3831B.

As further depicted in FIGS. 23 and 24B, the eighth half-bridge circuit 3831H can include the thirteenth strain gauge resistor (R₁₃) and the fourteenth strain gauge resistor (R₁₄). As depicted in FIG. 24B, the thirteenth and fourteenth strain gauge resistors (R₁₃, R₁₄) are positioned in axial alignment with the beam center axis (A_{B}). In some embodiments, a portion of the thirteenth strain gauge resistor (R₁₃) is positioned axially between the portions of the fourteenth strain gauge resistor (R₁₄), and a portion of the fourteenth strain gauge resistor (R₁₄) is positioned axially between the portions of the thirteenth strain gauge resistor (R₁₃). As depicted, one of the thirteenth and fourteenth strain gauge resistors (R₃, R₁₄) is a tension strain gauge resistor while the other is a compression strain gauge resistor. The eighth half-bridge circuit 3831H is positioned distally relative to the fourth half-bridge circuit 3831D.

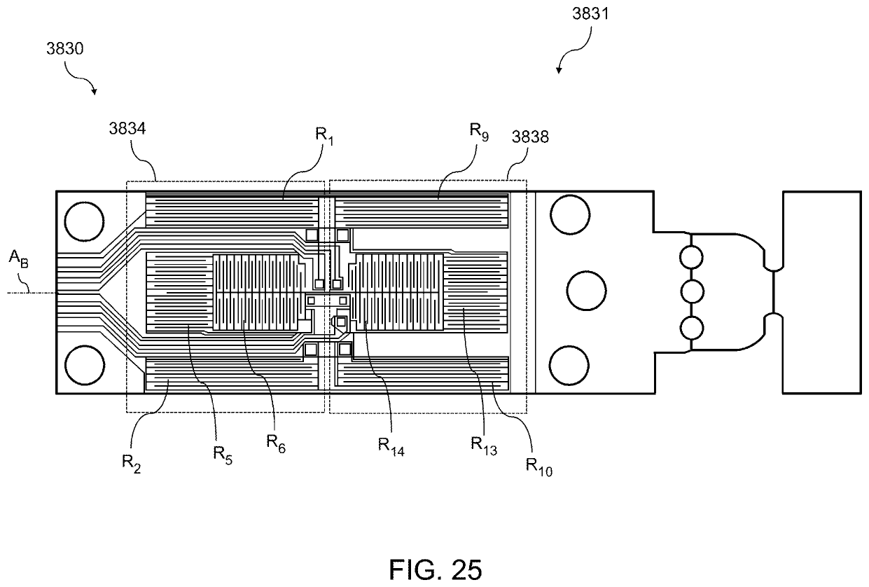

FIG. 25 is an enlarged illustration of a distal portion of the force sensor unit 3800 illustrating an alternative arrangement of the strain gauge resistors to that depicted in FIG. 24B. As depicted in FIG. 25, the first and second strain gauge resistors (R₁, R₂) of the second half-bridge circuit 3831B can be positioned on opposite sides of the beam center axis (A_{B}) and equidistant from the center axis (A_{B}). For example, the first and second strain gauge resistors (R₁, R₂) can be positioned equidistant between the beam center axis (A_{B}) and a side edge of the surface to which they are mounted. In some embodiments, the first and second strain gauge resistors (R₁, R₂) can be positioned at the same proximal position along the beam center axis (A_{B}). As depicted, in some embodiments, the first and second strain gauge resistors (R₁, R₂) are both the same type of strain gauge resistor (e.g., are both tension strain gauge resistors). As further depicted in FIG. 25, the fifth and sixth strain gauge resistors (R₅, R₆) of the fourth half-bridge circuit 3831D are positioned in axial alignment with the beam center axis (A_{B}). As depicted, the sixth strain gauge resistor (R₆) is positioned distally relative to the fifth strain gauge resistor (R₅). As depicted, the fifth strain gauge resistors (R₅) is a tension strain gauge resistor while the sixth strain gauge resistor (R₆) is a compression strain gauge resistor.

As further depicted in FIG. 25, the ninth and tenth strain gauge resistors (R₉, R₁₀) of the sixth half-bridge circuit 3831F can be positioned on opposite sides of the beam center axis (A_{B}) and equidistant from the center axis (A_{B}). For example, the ninth and tenth strain gauge resistors (R₉, R₁₀) can be positioned equidistant between the beam center axis (A_{B}) and a side edge of the surface to which they are mounted. In some embodiments, the ninth and tenth strain gauge resistors (R₉, R₁₀) can be positioned at the same proximal position along the beam center axis (A_{B}). As depicted, in some embodiments, the ninth and tenth strain gauge resistors (R₉, R₁₀) are both the same type of strain gauge resistor (e.g., are both tension strain gauge resistors). The sixth half-bridge circuit 3831F is positioned distally relative to the second half-bridge circuit 3831B. Additionally, the thirteenth and fourteenth strain gauge resistors (R₁₃, R₁₄) of the eighth half-bridge circuit 3831H are positioned in axial alignment with the beam center axis (A_{B}). As depicted, the thirteenth strain gauge resistor (R₁₃) is positioned distally relative to the fourteenth strain gauge resistor (R₁₄). As depicted, the thirteenth strain gauge resistors (R₁₃) is a tension strain gauge resistor while the fourteenth strain gauge resistor (R₁₄) is a compression strain gauge resistor. The eighth half-bridge circuit 3831H is positioned distally relative to the fourth half-bridge circuit 3831D.

Although FIG. 25 is described with reference to the primary distal bridge-circuit combination 3834 and the secondary distal bridge-circuit combination 3838, the strain gauge resistors of the primary proximal bridge-circuit combination 3832 and the secondary proximal bridge-circuit combination 3836 may be similarly arranged in the described alternative arrangement.

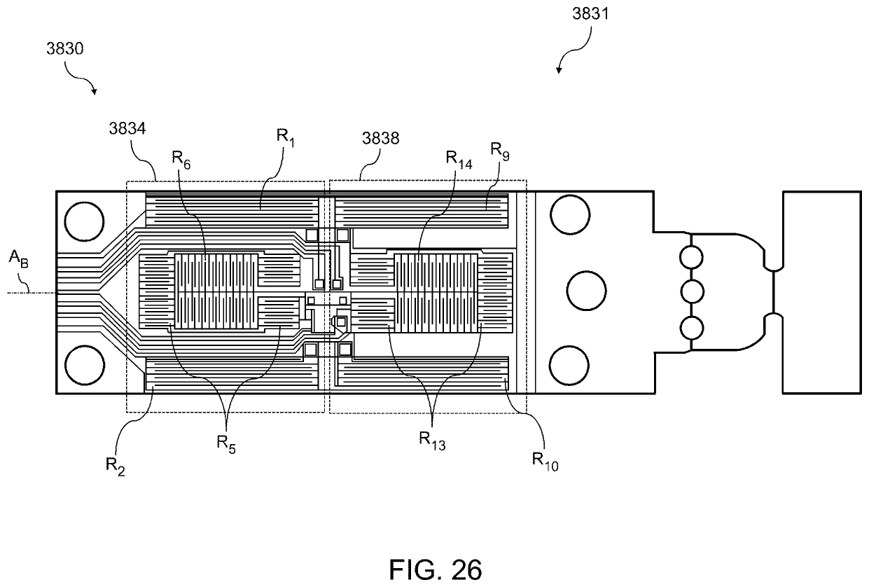

FIG. 26 is an enlarged illustration of a distal portion of the force sensor unit 3800 illustrating an alternative arrangement of the strain gauge resistors to that depicted in FIG. 24B. As depicted in FIG. 26, the first and second strain gauge resistors (R₁, R₂) of the second half-bridge circuit 3831B can be positioned on opposite sides of the beam center axis (A_{B}) and equidistant from the center axis (A_{B}). For example, the first and second strain gauge resistors (R₁, R₂) can be positioned equidistant between the beam center axis (As) and a side edge of the surface to which they are mounted. In some embodiments, the first and second strain gauge resistors (R₁, R₂) can be positioned at the same proximal position along the beam center axis (A_{B}). As depicted, in some embodiments, the first and second strain gauge resistors (R₁, R₂) are both the same type of strain gauge resistor (e.g., are both tension strain gauge resistors). As further depicted in FIG. 26, the fifth and sixth strain gauge resistors (R₅, R₆) of the fourth half-bridge circuit 3831D are positioned in axial alignment with the beam center axis (A_{B}). As depicted, the sixth strain gauge resistor (R₆) is positioned between the portions of the fifth strain gauge resistor (R₅). As depicted, the fifth strain gauge resistors (R₅) is a tension strain gauge resistor while the sixth strain gauge resistor (R₆) is a compression strain gauge resistor.

As further depicted in FIG. 26, the ninth and tenth strain gauge resistors (R₉, R₁₀) of the sixth half-bridge circuit 3831F can be positioned on opposite sides of the beam center axis (A_{B}) and equidistant from the center axis (A_{B}). For example, the ninth and tenth strain gauge resistors (R₉, R₁₀) can be positioned equidistant between the beam center axis (A_{B}) and a side edge of the surface to which they are mounted. In some embodiments, the ninth and tenth strain gauge resistors (R₉, R₁₀) can be positioned at the same proximal position along the beam center axis (A_{B}). As depicted, in some embodiments, the ninth and tenth strain gauge resistors (R₉, R₁₀) are both the same type of strain gauge resistor (e.g., are both tension strain gauge resistors). The sixth half-bridge circuit 3831F is positioned distally relative to the second half-bridge circuit 3831B. Additionally, the thirteenth and fourteenth strain gauge resistors (R₁₃, R₁₄) of the eighth half-bridge circuit 3831H are positioned in axial alignment with the beam center axis (A_{B}). As depicted, the fourteenth strain gauge resistor (R₁₄) is positioned between portions of the thirteenth strain gauge resistor (R₁₃). As depicted, the thirteenth strain gauge resistors (R₁₃) is a tension strain gauge resistor while the fourteenth strain gauge resistor (R₁₄) is a compression strain gauge resistor. The eighth half-bridge circuit 3831H is positioned distally relative to the fourth half-bridge circuit 3831D.

Although FIG. 26 is described with reference to the primary distal bridge-circuit combination 3834 and the secondary distal bridge-circuit combination 3838, the strain gauge resistors of the primary proximal bridge-circuit combination 3832 and the secondary proximal bridge-circuit combination 3836 may be similarly arranged in the described alternative arrangement.

As shown particularly in FIG. 27, a schematic diagram of one embodiment of suitable components that may be included within the controller 1800 is illustrated. In some embodiments, the controller 1800 is positioned within a component of the surgical system 1000, such as the user control unit 1100 and/or the optional auxiliary equipment unit 1150. However, the controller 1800 may also include distributed computing systems wherein at least one aspect of the controller 1800 is at a location which differs from the remaining components of the surgical system 1000 for example, at least a portion of the controller 1800 may be an online controller.

As depicted, the controller 1800 includes one or more processor(s) 1802 and associated memory device(s) 1804 configured to perform a variety of computer implemented functions (e.g., performing the methods, steps, calculations and the like and storing relevant data as disclosed herein). Additionally, in some embodiments, the controller 1800 includes a communication module 1806 to facilitate communications between the controller 1800 and the various components of the surgical system 1000.

As used herein, the term “processor” refers not only to integrated circuits referred to in the art as being included in a computer, but also refers to a controller, a microcontroller, a microcomputer, a programmable logic controller (PLC), an application specific integrated circuit, and other programmable circuits. Additionally, the memory device(s) 1804 may generally comprise memory element(s) including, but not limited to, computer readable medium (e.g., random access memory (RAM)), computer readable nonvolatile medium (e.g., a flash memory), a floppy disk, a compact disc read only memory (CD ROM), a magneto optical disk (MOD), a digital versatile disc (DVD) and/or other suitable memory elements. Such memory device(s) 1804 may generally be configured to store suitable computer readable instructions that, when implemented by the processor(s) 1802, configure the controller 1800 to perform various functions.

In some embodiments, the controller 1800 includes a haptic feedback module 1820. The haptic feedback module 1820 may be configured to deliver a haptic feedback to the operator (S) based on inputs received from a force sensor unit of the instrument 1400 (e.g., the force sensor unit 3800, including the strain sensors 3830 (FIG. 13). In some embodiments, haptic feedback module 1820 may be an independent module of the controller 1800. However, in some embodiments the haptic feedback module 1820 may be included within the memory device(s) 1804.

The communication module 1806 may include a control input module 1808 configured to receive control inputs from the operator/surgeon (S), such as via the input device 1116 of the user control unit 1100. The communication module may also include an indicator module 1812 configured to generate various indications in order to alert the operator (S).

The communication module 1806 may also include a sensor interface 1810 (e.g., one or more analog to digital converters) to permit signals transmitted from one or more sensors (e.g., strain sensors 2830 of the force sensor unit 2800 (FIG. 12) to be converted into signals that can be understood and processed by the processors 1802. The sensors may be communicatively coupled to the communication module 1806 using any suitable means. For example the sensors may be coupled to the communication module 1806 via a wired connection and/or via a wireless connection, such as by using any suitable wireless communications protocol known in the art. Additionally, in some embodiments, the communication module 1806 includes a device control module 1814 configured to modify an operating state of the instrument 1400 (and/or any of the instruments described herein (e.g., 2400, 3400). Accordingly, the communication module is communicatively coupled to the manipulator 1200 and/or the instrument 1400. For example, the communications module 1806 may communicate to the manipulator 1200 and/or the instrument 1400 an excitation voltage for the strain sensor(s), a handshake and/or excitation voltage for a positional sensor (e.g., for detecting the position of the designated portion relative to the cannula), cautery controls, positional setpoints, and/or an end effector operational setpoint (e.g., gripping, cutting, and/or other similar operation performed by the end effector).

FIG. 28 is a flow chart of a method 4000 of control for a surgical system according to an embodiment. The method 4000 may, in an embodiment, be performed via a teleoperated system, such as system 1000 as described with reference to FIGS. 4-27. However, it should be appreciated that in various embodiments, aspects of the method 4000 may be accomplished via additional embodiments of the system 1000 or components thereof, such as instrument 2400 and or instrument 3400 as described herein. Accordingly, the method 4000 may be implemented on any suitable device as described herein. Thus, the method 4000 is described below with reference to medical instrument 2400 and the controller 1800 of the system 1000 as previously described, but it should be understood that the method 4000 can be employed using any of the medical devices/instruments and controllers described herein.

As depicted at 4002 in FIG. 28, controller provides haptic feedback to the input device of the surgical system. As depicted at 4004, the controller determines a moment at a reference location of the distal end portion of the instrument. As depicted at 4006, the controller then determines a deflection of the reference location based on the moment (and force). On a first condition in which the deflection is greater than a deflection threshold, the controller, as depicted at 4008, provides an indication to an operator of the input device that a restriction of the haptic feedback is provided to, or is available to be provided to, the input device.

While various embodiments have been described above, it should be understood that they have been presented by way of example only, and not limitation. Where methods and/or schematics described above indicate certain events and/or flow patterns occurring in certain order, the ordering of certain events and/or operations may be modified. While the embodiments have been particularly shown and described, it will be understood that various changes in form and details may be made.

For example, any of the instruments described herein (and the components therein) are optionally parts of a surgical assembly that performs minimally invasive surgical procedures, and which can include a manipulator unit, a series of kinematic linkages, a series of cannulas, or the like. Thus, any of the instruments described herein can be used in any suitable surgical system, such as the MIRS system 1000 shown and described above. Moreover, any of the instruments shown and described herein can be used to manipulate target tissue during a surgical procedure. Such target tissue can be cancer cells, tumor cells, lesions, vascular occlusions, thrombosis, calculi, uterine fibroids, bone metastases, adenomyosis, or any other bodily tissue. The presented examples of target tissue are not an exhaustive list. Moreover, a target structure can also include an artificial substance (or non-tissue) within or associated with a body, such as for example, a stent, a portion of an artificial tube, a fastener within the body or the like.

For example, any of the components of a surgical instrument as described herein can be constructed from any material, such as medical grade stainless steel, nickel alloys, titanium alloys or the like. Further, any of the links, tool members, beams, shafts, cables, or other components described herein can be constructed from multiple pieces that are later joined together. For example, in some embodiments, a link can be constructed by joining together separately constructed components. In other embodiments however, any of the links, tool members, beams, shafts, cables, or components described herein can be monolithically constructed.

Although various embodiments have been described as having particular features and/or combinations of components, other embodiments are possible having a combination of any features and/or components from any of embodiments as discussed above. Aspects have been described in the general context of medical devices, and more specifically surgical instruments, but inventive aspects are not necessarily limited to use in medical devices.

1. A method of control for a surgical system, the surgical system including a controller, an input device, and a medical instrument operably coupled to be controlled by the input device via the controller, the method comprising:
providing, via the controller, haptic feedback to the input device;
determining, via the controller, a moment at a reference location of a distal end portion of the instrument;
determining, via the controller, a deflection of the reference location based on the moment; and
on a first condition in which the deflection is greater than a deflection threshold, providing, via the controller, an indication to an operator of the input device that a restriction of the haptic feedback is provided to, or is available to be provided to, the input device.

2. The method of claim 1, wherein:
the reference location is located at a longitudinal position along the medical instrument that is coplanar with a hard stop location that limits deflection of the reference location.

3. The method of claim 1, wherein:
the medical instrument includes a force sensor unit and an end effector coupled to the distal end portion of the instrument;
the force sensor unit includes a beam and one or more strain sensors coupled to the beam; and
determining the moment includes determining the moment based on output of the one or more strain sensors.

4. The method of claim 3, wherein:
the one or more strain sensors includes a first bridge circuit and a second bridge circuit;
the first bridge circuit includes a first gauge resistor and a second gauge resistor; and
the second bridge circuit includes a third gauge resistor and a fourth gauge resistor.

5. The method of claim 3, wherein:
the one or more strain sensors includes a first bridge circuit and a second bridge circuit; and
determining the moment is based on an output voltage of the first bridge circuit and an output voltage of the second bridge circuit.

6. The method of claim 5, wherein:
the method further includes determining, via the controller, a determined force at the reference location based on the output voltage of the first bridge circuit and the output voltage of the second bridge circuit.

7. The method of claim 1, wherein:
the method further includes determining, via the controller, an estimated applied force on the medical instrument based on the moment; and
the method further includes executing, by the controller, an operation of the surgical system based on the estimated applied force.

8. The method of claim 7, wherein:
the method further includes delivering a haptic feedback magnitude to an operator of the input device corresponding to the estimated applied force.

9. The method of claim 7, wherein:
the method further includes halting, via the controller, an operation of the surgical system when the estimated applied force exceeds a threshold value.

10. The method of claim 1, wherein:
the indication includes one or more of a visual indication, an audible indication, or a haptic indication.

11. The method of claim 1, wherein:
the restriction of the haptic feedback corresponds to a halting of an operation of the surgical system.

12. The method of claim 1, wherein:
subsequent to the first condition and on a second condition in which the deflection is less than the deflection threshold, the method includes removing, via the controller, the restriction of the haptic feedback.

13. A surgical system, comprising:
a medical instrument supported by a manipulator unit;
an input device operably coupled to the medical instrument; and
a controller operably coupled to the manipulator unit and the input device, the controller comprising at least one processor and a haptic feedback module configured to perform a plurality of operations, the plurality of operations comprising:
providing haptic feedback to the input device,
determining a moment at a reference location of a distal end portion of the instrument,
determining a deflection of the reference location based on the moment, and
on a first condition in which the deflection is greater than a deflection threshold, providing an indication to an operator of the input device that a restriction of the haptic feedback is provided to, or is available to be provided to, the input device.

14. The surgical system of claim 13, wherein:
the reference location is located at a longitudinal position along the medical instrument that is coplanar with a hard stop positioned location that limits deflection of the reference location.

15. The surgical system of claim 13, wherein:
the medical instrument includes a force sensor unit and an end effector coupled to the distal end portion of the instrument;
the force sensor unit includes a beam and one or more strain sensors coupled to the beam; and
determining the moment includes determining the moment based on output of the one or more strain sensors.

16. The surgical system of claim 15, wherein:
the one or more strain sensors includes a first bridge circuit and a second bridge circuit;
the first bridge circuit includes a first gauge resistor and a second gauge resistor; and
the second bridge circuit includes a third gauge resistor and a fourth gauge resistor.

17. The surgical system of claim 15, wherein:
the one or more strain sensors includes a first bridge circuit and a second bridge circuit; and
determining the moment is based on an output voltage of the first bridge circuit and an output voltage of the second bridge circuit.

18. The surgical system of claim 17, wherein:
the plurality of operations further includes determining a determined force at the reference location based on the output voltage of the first bridge circuit and the output voltage of the second bridge circuit.

19. The surgical system of claim 13, wherein:
the plurality of operations further includes:
determining an estimated applied force on the medical instrument based on the moment, and
executing an operation of the surgical system based on the estimated applied force.

20. The surgical system of claim 19, wherein:
the plurality of operations further includes delivering a haptic feedback magnitude to an operator of the input device corresponding to the estimated applied force.

21-24. (canceled)
